SEQUENCE LISTING (33 sequences)

SEQ ID NO: 1 - protein, 90 aa, Artificial Sequence
  FRB domain
MEMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 2 - protein, 90 aa, Artificial Sequence
  FRB domain
MEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 3 - protein, 353 aa, Artificial Sequence
  MND Promoter
GAACAGAGAAACAGGAGAATATGGGCCAAACAGGATATCTGTGGTAAGCAGTTCCTGCCCCGGCTCAGGGCCAAGAACAGTTGGAACAGCAGAATATGGGCCAAACAGGATATCTGTGGTAAGCAGTTCCTGCCCCGGCTCAGGGCCAAGAACAGATGGTCCCCAGATGCGGTCCCGCCCTCAGCAGTTTCTAGAGAACCATCAGATGTTTCCAGGGTGCCCCAAGGACCTGAAATGACCCTGTGCCTTATTTGAACTAACCAATCAGTTCGCTTCTCGCTTCTGTTCGCGCGCTTCTGCTCCCCGAGCTCTATATAAGCAGAGCTCGTTTAGTGAACCGTCAGATCGCTAGC

SEQ ID NO: 4 - protein, 251 aa, Artificial Sequence
  IL2Rgamma complex
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 429 aa, Artificial Sequence
  IL2Rbeta cmplex
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 108 aa, Artificial Sequence
  FKBP domain
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGE

SEQ ID NO: 7-17 - skipped (11 entries)

SEQ ID NO: 18 - protein, 153 aa, Artificial Sequence
  IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 19 - protein, 177 aa, Artificial Sequence
  IL7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 162 aa, Artificial Sequence
  IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 21 - protein, 271 aa, Artificial Sequence
  membrane IL7
MAHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 22 - protein, 256 aa, Artificial Sequence
  membrane IL-15
MGLVRRGARAGPRMPRGWTALCLLSLLPSGFMAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 23 - protein, 251 aa, Artificial Sequence; identical to SEQ ID NO: 4
  IL2Rgamma complex

SEQ ID NO: 24 - protein, 251 aa, Artificial Sequence; identical to SEQ ID NO: 4
  IL2Rgamma complex

SEQ ID NO: 25 - protein, 251 aa, Artificial Sequence; identical to SEQ ID NO: 4
  IL2Rgamma complex

SEQ ID NO: 26 - protein, 429 aa, Artificial Sequence; identical to SEQ ID NO: 5
  IL2Ebeta complex

SEQ ID NO: 27 - protein, 429 aa, Artificial Sequence; identical to SEQ ID NO: 5
  IL2Rbeta complex

SEQ ID NO: 28 - protein, 429 aa, Artificial Sequence; identical to SEQ ID NO: 5
  IL2Rbeta complex

SEQ ID NO: 29 - protein, 429 aa, Artificial Sequence; identical to SEQ ID NO: 5
  IL7Ralpha complex

SEQ ID NO: 30 - protein, 4 aa, Artificial Sequence
  glycine spacer
GGGS

SEQ ID NO: 31 - protein, 7 aa, Artificial Sequence
  glycine spacer
GGGSGGG

SEQ ID NO: 32 - protein, 3 aa, Artificial Sequence
  glycine spacer
GGG

SEQ ID NO: 33 - protein, 429 aa, Artificial Sequence; identical to SEQ ID NO: 5
  IL2Ralpha complex

(19) United States
(12) Patent Application Publication
(10) Pub. No.: US 2023/0407330 A1
(43) Pub. Date: Dec. 21, 2023

(54) VECTOR SYSTEM FOR DELIVERY OF MULTIPLE POLYNUCLEOTIDES AND USES THEREOF

(71) Applicant: Umoja Biopharma, Inc., Seattle, WA (US)

(72) Inventors: Andrew SCHARENBERG, Seattle, WA (US); Laurie BEITZ, Seattle, WA (US)

(73) Assignee: Umoja Biopharma, Inc., Seattle, WA (US)

(21) Appl. No.: 18/037,986

(22) PCT Filed: Nov. 18, 2021
(86) PCT No.: PCT/US2021/059931

§ 371 Date: May 19, 2023

Related U.S. Application Data
(60) Provisional application No. 63/116,611, filed on Nov. 20, 2020.

(51) Int. Cl. C12N 15/86; C12N 9/90; C07K 14/715

(52) U.S. Cl. CPC C12N 15/86; C12Y 502/01008; C12N 9/90; C07K 14/7155; C12N 2740/15043

(57) ABSTRACT

The present disclosure relates to a vector system comprising at least two polynucleotides, each polynucleotide may comprise a polynucleotide sequence encoding a polypeptide component of a macromolecular complex. Assembly of the macromolecular complex in a cell transduced with the polynucleotides may promote growth and/or survival of a cell.

### RELATED APPLICATIONS

This application claims priority to, and the benefit of, U.S. Provisional Application No. 63/116,611, filed Nov. 20, 2020. The contents of the aforementioned patent application are incorporated herein by reference in its entirety.

### TECHNICAL FIELD

The present disclosure relates generally to a viral vector system encoding components of a macromolecular complex, compositions comprising, and methods of use thereof.

### SEQUENCE LISTING

The instant application contains a Sequence Listing which has been submitted in ASCII format via EFS-Web and is hereby incorporated by reference in its entirety. Said ASCII copy, created on Nov. 17, 2021, is named “UMOJ-008-01WO_SeqList_ST25.txt” and is 47 KB in size.

### BACKGROUND

Many vector systems for delivery of polynucleotides into cells have upper limits on the size of the polynucleotide to be delivered. For example, AAV vectors have a packaging limit of about ˜5 kb. Lentiviral vectors have a packaging limit of about ˜10 kB. To deliver larger genes, a variety of technologies have been developed. Known methods generally rely on the interaction of polynucleotides in the cell, e.g., homologous recombination or trans-splicing. For example, Tornabene, P. et al. (2020) Human Gene Therapy 31(47-56) discloses the use of multiple AAV vectors to deliver large genes. Zufferey, R. et al. (1998) Journal of Virology 72; 12(9873-9880) discloses the use of self-inactivating HIV-1 vectors for stable transgene expression with larger cloning capacity. Cockrell, A. S. and Kafri, T. (2007) Molecular Biotechnology 36(184-204) disclose the use of lentiviral vectors for transduction of nondividing cells and generation of transgenic animals.

There remains an unmet need for polynucleotide delivery technologies.

### SUMMARY

One aspect of the present disclosure provides a vector system comprising at least two polynucleotides, each polynucleotide comprising a polynucleotide sequence encoding a polypeptide component of a macromolecular complex, wherein assembly of the macromolecular complex in a cell transduced with the at least two polynucleotides promotes growth and/or survival of a cell.

In some embodiments, the vector system comprises a macromolecular complex that is a multipartite cell-surface receptor.

In some embodiments, the vector system comprises a single vector comprising two of the polynucleotides.

In some embodiments, the vector system comprises a single vector that is a single lentivirus vector.

In some embodiments, the vector system comprises two vectors, each vector comprising one of the polynucleotides.

In some embodiments, the vector system comprises vectors that are two lentivirus vectors.

In some embodiments, the assembly of the macromolecular complex is controlled by a ligand.

In some embodiments, the vector system comprises a first polynucleotide comprising a polynucleotide sequence encoding a first polypeptide component of the macromolecular complex comprising an FKBP-rapamycin complex binding domain (FRB domain) or a functional variant thereof, and a second polynucleotide comprising a polynucleotide sequence encoding a second polypeptide component of the macromolecular complex comprising an FK506 binding protein domain (FKBP) or a functional variant thereof; and/or wherein the ligand is rapamycin.

In some embodiments, the vector system comprises a FRB domain polypeptide that shares at least 75%, at least 80%, at least 85%, at least 90%, at least 95%, at least 99%, or 100% identity to SEQ ID NO: 1.

In some embodiments, the vector system comprises a FRB domain polypeptide that shares at least 75%, at least 80%, at least 85%, at least 90%, at least 95%, at least 99%, or 100% identity to SEQ ID NO: 2

In some embodiments, the vector system comprises a FKBP polypeptide that shares at least 75%, at least 80%, at least 85%, at least 90%, at least 95%, at least 99%, or 100% identity to SEQ ID NO: 6.

In some embodiments, expression of the macromolecular complex is under the control of an inducible genetic or biochemical system.

In some embodiments, each polynucleotide of the vector system is operatively linked to a promoter.

In some embodiments, the promoter is an inducible promoter.

In some embodiments, at least one of the polynucleotides comprises a polynucleotide sequence that confers resistance to an immunosuppressive agent.

In some embodiments, the polynucleotide sequence that confers resistance to an immunosuppressive agent encodes a polypeptide that binds rapamycin, wherein optionally, the polypeptide is FRB.

In some embodiments, at least one polynucleotide sequence is capable of transducing T cells, NK cells, or NKT cells.

In some embodiments, at least one polynucleotide sequence is capable of transducing T cells, NK cells, or NKT cells in vivo.

In some embodiments, at least one polynucleotide sequence is capable of transducing T cells, NK cells, or NKT cells in vitro.

In some embodiments, cells that have been transduced with both vector genomes are selectively selected. In some embodiments, transduction with both vector genomes promotes growth and/or survival of the transduced cell.

In some embodiments, the vector system comprises at least one retroviral particle,

wherein the retroviral particle comprises one or more transduction enhancers, wherein the transduction enhancer is selected from the group consisting of a T-cell activation receptor, a NK-cell activation receptor, and a co-stimulatory molecule.

In some embodiments, the one or more transduction enhancers comprise one or more of anti-CD3scFv, CD86, and CD137L.

In some embodiments, the first vector comprises a polynucleotide sequence encoding:
(a) a promoter;
(b) a FK506 binding protein (FKBP) domain or a portion thereof
(c) an IL-2 receptor transmembrane domain
(d) an interleukin-2 receptor subunit gamma (IL2Rγ) domain; and
(e) a first chimeric antigen receptor (CAR).

In some embodiments, the second vector comprises a polynucleotide sequence encoding:
(a) a promoter;
(b) FKBP rapamycin binding (FRB) domain or a portion thereof
(c) an IL-2 receptor transmembrane domain
(d) an interleukin-2 receptor subunit beta (IL2Rβ) domain; and
(e) a second CAR.

In some embodiments, the FKBP domain or a portion thereof and FRB domain or a portion thereof heterodimerize in the presence of rapamycin to promote growth and/or survival of a cell.

In some embodiments of the vector system, the promoter is MND.

In some embodiments, the MND promoter shares at least 75%, at least 80%, at least 85%, at least 90%, at least 95%, at least 99%, or 100% identity to SEQ ID NO: 3.

In some embodiments, the IL2Rβ domain polypeptide shares at least 75%, at least 80%, at least 85%, at least 90%, at least 95%, at least 99%, or 100% identity to SEQ ID NO: 4.

In some embodiments, the IL2Rβ domain polypeptide shares at least 75%, at least 80%, at least 85%, at least 90%, at least 95%, at least 99%, or 100% identity to SEQ ID NO: 5.

In some embodiments, the first CAR polypeptide comprises an antigen binding molecule that specifically binds to the cell surface antigen CD19.

In some embodiments, the second CAR polypeptide comprises an antigen binding molecule that specifically binds to the cell surface antigen CD20.

One aspect of the present disclosure provides a method comprising administering to a subject a vector system of any of the embodiments as described above.

Further aspects and embodiments of the invention are provided by the Detailed Description that follows.

### BRIEF DESCRIPTION OF THE DRAWINGS

FIG. 1 is a diagram depicting an embodiment of the dual vector system encoding two polynucleotide sequences each encoding a component of a macromolecular complex (RACRγ and RACRβ) which binds and confers resistance to rapamycin. The vector system may also encode a cytosolic FRB domain protein which additionally sequesters rapamycin through complexation with FKBP.

FIG. 2 is a diagram depicting embodiments of the dual vector system encoding two polynucleotide sequences each encoding a component of a macromolecular complex (RACRγ and RACRβ), a cytosolic FRB domain, and a CAR.

FIG. 3 depicts a vector map for pRRL-MND-Human-Frb-RACCRb-CD19_CAR-VTw

FIG. 4 depicts a vector map for pRRL-MND-human-Frb-RACCRg-CD20_CAR-VTw

FIGS. 5A-5B depict graphs of lentiviral particle titers. For the supernatant samples (FIG. 5A), the lentviral titer was 3.65×10⁵ TU/ml, and for the concentrated samples (FIG. 5B), the lentviral titer was 1.12×10⁸ TU/ml.

FIGS. 6A-6B are flow cytometry staining plots depicting surface expression of CD19 and CD20 CARs in transduced T-cells. FIG. 6A depicts a flow cytometry staining plot of dual vector system transduced cells that were not stimulated with rapamycin. FIG. 6B depicts a flow cytometry staining plot of cells transduced with a dual vector system and stimulated with 10 mM rapamycin.

FIGS. 7A-7D are flow cytometry staining plots depicting CAR T cells co-cultured with tumor cells. CD19 negative/CD20 negative K562 tumor cells remained unaffected in the absence (FIG. 7A) or presence (FIG. 7B) of dual vector system transduced T cells. CD19 positive/CD20 negative K562 KI tumor cells were unaffected in the absence (FIG. 7C) of dual vector system transduced T cells, while cells transduced with the dual vector system eradicated CD19 positive/CD20 negative tumor cells (FIG. 7D).

FIGS. 8A-8B are flow cytometry staining plots depicting T cells expressing a CD20 CAR co-cultured with CD19 KO/CD20+ RAJI tumor cells. CD19 KO/CD20+ RAJI tumor cells were unaffected by untransduced T cells (FIG. 8A), while cells transduced with the dual vector system eradicated CD19 negative/CD20 positive RAJI tumor cells (FIG. 8B).

FIG. 9 is a graph depicting IFNγ cytokine production in response to dual vector system transduced T cells in control (target cells only), non-transduced T cells (No CAR), and transduced T cells (DP CAR) following 68 hours of co-culture with control cells (no target), K562 cells (no surface antigen), K562 CD19 knock-in (KI) cells (K562+19), RAJI CD19 knock-out (KO) cells (Raji-19), or RAJI CD19+/CD20+(Raji) cells.

FIG. 10 is a graph depicting IL-2 cytokine production in response to dual vector system transduced T cells in control (target cells only), non-transduced T cells (No CAR), and transduced T cells (DP CAR) following 68 hours of co-culture with control cells (no target), K562 cells (no surface antigen), K562 CD19 knock-in (KI) cells (K562+19), RAJI CD19 knock-out (KO) cells (Raji-19), or RAJI CD19+/CD20+(Raji) cells.

FIG. 11 is a graph depicting TNFα cytokine production in response to dual vector system transduced T cells in control (target cells only), non-transduced T cells (No CAR), and transduced T cells (DP CAR) following 68 hours of co-culture with control cells (no target), K562 cells (no surface antigen), K562 CD19 knock-in (KI) cells (K562+19), RAJI CD19 knock-out (KO) cells (Raji-19), or RAJI CD19+/CD20+(Raji) cells.

FIG. 12 is a graph depicting IL-13 cytokine production in response to dual vector system transduced T cells in control (target cells only), non-transduced T cells (No CAR), and transduced T cells (DP CAR) following 68 hours of co-culture with control cells (no target), K562 cells (no surface antigen), K562 CD19 knock-in (KI) cells (K562+19), RAJI CD19 knock-out (KO) cells (Raji-19), or RAJI CD19+/CD20+(Raji) cells.

FIGS. 13A-13C are flow cytometry staining plots depicting dual CAR T cell enrichment following rapamycin stimulation. Surface expression of both CD19 and CD20 CARs was analyzed using FITC-CD19 antigen and PE-CD20 antigen. Dual vector system transduced T cells were analyzed pre-stimulation (FIG. 13A), following co-culture with K562 cells not expressing antigen (FIG. 13B), and following co-culture with K562 cells expressing CD19 (FIG. 13C).

FIG. 14 is a graph depicting the expansion of dual vector system transduced T cells in response to RAJI target cell co-culture for 7 days. Cell number was analyzed as a function of transduced effector T cell: RAJI target cell ratios.

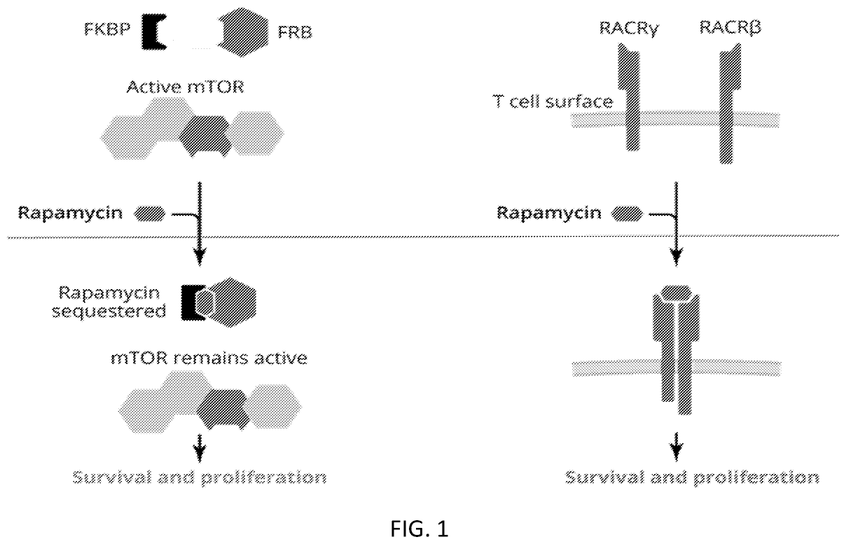

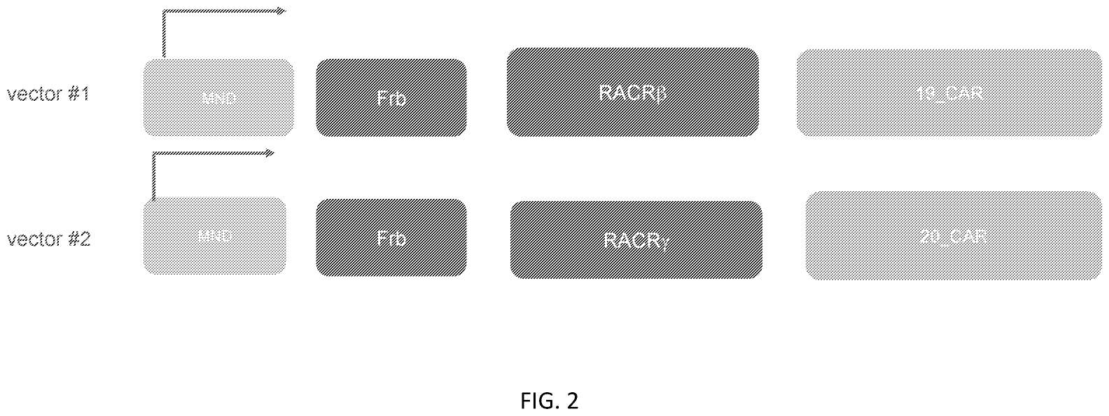

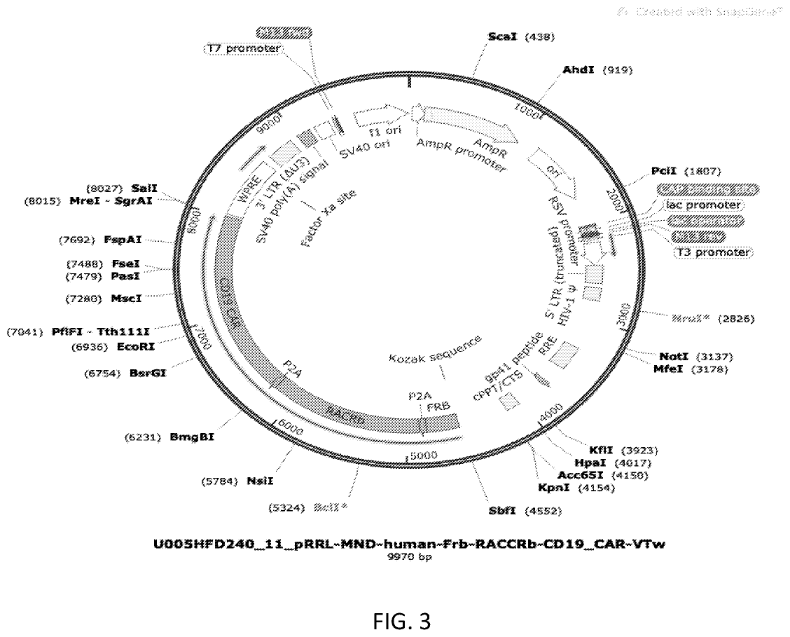

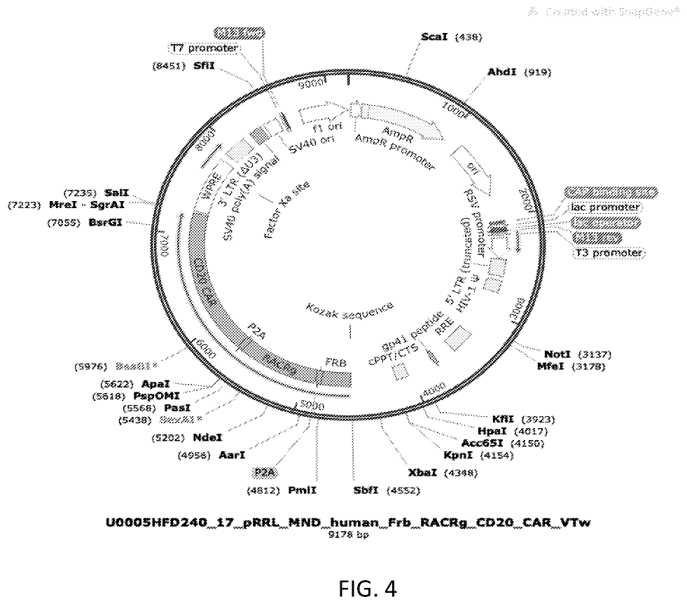

### DETAILED DESCRIPTION

The disclosure relates generally to a vector system comprising at least two polynucleotides, each polynucleotide comprising a polynucleotide sequence encoding a polypeptide component of a macromolecular complex, wherein assembly of the macromolecular complex in a cell transduced with the at least two polynucleotides promotes growth and/or survival of a cell.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

As used in the description of the invention and the appended claims, the singular forms “a”, “an”, and “the” are intended to include the plural forms as well, unless the context clearly indicates otherwise.

“Subject” as used herein includes is a mammal, such as primate, mouse, rat, dog, cat, cow, horse, goat, camel, sheep or a pig, preferably a human.

“Treat,” “treating” or “treatment” as used herein also refers to any type of action or administration that imparts a benefit to a subject that has a disease or disorder, including improvement in the condition of the patient (e.g., reduction or amelioration of one or more symptoms), healing, etc.

Also as used herein, “and/or” refers to and encompasses any and all possible combinations of one or more of the associated listed items, as well as the lack of combinations when interpreted in the alternative (or).

Unless the context indicates otherwise, it is specifically intended that the various features described herein can be used in any combination. Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed.

It will also be understood that, as used herein, the terms example, exemplary, and grammatical variations thereof are intended to refer to non-limiting examples and/or variant embodiments discussed herein, and are not intended to indicate preference for one or more embodiments discussed herein compared to one or more other embodiments.

All publications, patent applications, patents and other references cited herein are incorporated by reference in their entireties for the teachings relevant to the sentence and/or paragraph in which the reference is presented.

Unless the context indicates otherwise, it is specifically intended that the various features described herein can be used in any combination.

Moreover, the present disclosure also contemplates that in some embodiments, any feature or combination of features set forth herein can be excluded or omitted.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence. The nucleic acid may produce a polypeptide which comprises one or more sequences encoding a mitogenic transduction enhancer and/or one or more sequences encoding a cytokine-based transduction enhancer. The cleavage site may be self-cleaving, such that when the polypeptide is produced, it is immediately cleaved into the receptor component and the signaling component without the need for any external cleavage activity.

### EMBODIMENTS

One aspect of the present disclosure provides a vector system comprising at least two polynucleotides, each polynucleotide comprising a polynucleotide sequence encoding a polypeptide component of a macromolecular complex, wherein assembly of the macromolecular complex in a cell transduced with the at least two polynucleotides promotes growth and/or survival of a cell.

In some embodiments, the vector system comprises a macromolecular complex that is a multipartite cell-surface receptor.

In some embodiments, the multipartite cell-surface receptor is a proliferatory receptor.

In some embodiments, the proliferatory receptor, optionally induced by a ligand, is delivered to a cell on two different polynucleotides.

In some embodiments, the vector system comprises a single vector comprising two of the polynucleotides.

In some embodiments, the vector system comprises a single vector that is a single lentivirus vector.

In some embodiments, the vector system comprises two vectors, each vector comprising one of the polynucleotides.

In some embodiments, the vector system comprises two vectors that are two lentivirus vectors.

In some embodiments, the vector system comprises at least two polynucleotides and each polynucleotide is encased in a separate capsid.

In some embodiments, the at least two polynucleotides are co-packaged in a single lentiviral particle. In some embodiments, the at least two polynucleotides are packaged into at least two lentiviral particles.

In some embodiments, two lentiviral genomes are transduced into and integrated in the same cell.

In some embodiments, the assembly of the macromolecular complex is controlled by a ligand.

In some embodiments, the ligand is rapamycin.

In some embodiments, the ligand is a protein, an antibody, a small molecule, or a drug. In some embodiments, the ligand is rapamycin or a rapamycin analog (rapalogs). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Thus, in some embodiments, the rapalog is everolimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophernolic acid, benidipine hydrochloride, rapamine, AP23573, or AP1903, or metabolites, derivatives, and/or combinations thereof. In some embodiments, the ligand is an IMID-class drug (e.g. thalidomide, pomalidimide, lenalidomide or related analogues).

In some embodiments, the molecule is selected from FK1012, tacrolimus (FK506), FKCsA, rapamycin, coumermycin, gibberellin, HaXS, TMP-HTag, and ABT-737 or functional derivatives thereof.

In some embodiments, the vector system comprises a first polynucleotide comprising a polynucleotide sequence encoding a first polypeptide component of the macromolecular complex comprising an FKBP-rapamycin complex binding domain (FRB domain) or a functional variant thereof.

In some embodiments, the vector system comprises a second polynucleotide comprising a polynucleotide sequence encoding a second polypeptide component of the macromolecular complex comprising an FK506 binding protein domain (FKBP) or a functional variant thereof.

In some embodiments, the vector system comprises a first polynucleotide comprising a polynucleotide sequence encoding a first polypeptide component of the macromolecular complex comprising an FKBP-rapamycin complex binding domain (FRB domain) or a functional variant thereof, and a second polynucleotide comprising a polynucleotide sequence encoding a second polypeptide component of the macromolecular complex comprising an FK506 binding protein domain (FKBP) or a functional variant thereof.

In some embodiments, the vector system comprises a FRB domain polypeptide that shares at least 75%, at least 80%, at least 85%, at least 90%, at least 95%, at least 99%, or 100% identity to SEQ ID NO: 1.

(SEQ ID NO: 1)
MEMWHEGLEEASRLYFGERNVKGMFEVLEPLHAMMERGPQTLKETS
FNQAYGRDLMEAQEWCRKYMKSGNVKDLTQAWDLYYHVFRRISK

In some embodiments, the vector system comprises a FRB domain polypeptide that shares at least 75%, at least 80%, at least 85%, at least 90%, at least 95%, at least 99%, or 100% identity to SEQ ID NO: 2

(SEQ ID NO: 2)
MEMWHEGLEEASRLYFGERNVKGMFEVLEPLHAMMERGPQTLKETS
FNQAYGRDLMEAQEWCRKYMKSGNVKDLLQAWDLYYHVFRRISK

In some embodiments, the vector system comprises a FKBP polypeptide that shares at least 75%, at least 80%, at least 85%, at least 90%, at least 95%, at least 99%, or 100% identity to SEQ ID NO: 6.

(SEQ ID NO: 6)
GVQVETISPGDGRTFPKRGQTCVVHYTGMLEDGKKFDSSRDRNKPFK
FMLGKQEVIRGWEEGVAQMSVGQRAKLTISPDYAYGATGHPGIIPPHATL
VFDVELLKLGE

In some embodiments, at least one of the polynucleotides comprises a polynucleotide sequence that confers resistance to an immunosuppressive agent.

In some embodiments, the polynucleotide sequence that confers resistance to an immunosuppressive agent encodes a polypeptide that binds rapamycin, wherein optionally, the polypeptide is FRB.

In some embodiments, at least one of the polynucleotides of the vector system comprises a cytosolic FRB domain.

In some embodiments, the FRB domain or a portion thereof and FKBP or a portion thereof form a complex that sequesters rapamycin in the transduced cell.

In some embodiments, the FKBP domain or a portion thereof and FRB domain or a portion thereof heterodimerize in the presence of rapamycin to promote growth and/or survival of a cell.

In some embodiments, expression of the macromolecular complex is under the control of an inducible genetic or biochemical system.

In some embodiments, each polynucleotide of the vector system is operatively linked to a promoter.

In some embodiments, the promoter is an inducible promoter.

In some embodiments, the retroviral particles and/or lentiviral particles of the disclosure comprise a polynucleotide comprising a sequence encoding a receptor that specifically binds to the ligand. In some embodiments, a sequence encoding a receptor that specifically binds to the ligand is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments of the vector system, the promoter is MND.

In some embodiments, the MND promoter shares at least 75%, at least 80%, at least 85%, at least 90%, at least 95%, at least 99%, or 100% identity to SEQ ID NO: 3.

(SEQ ID NO: 3)
GAACAGAGAAACAGGAGAATATGGGCCAAACAGGATATCTGTGGT
AAGCAGTTCCTGCCCCGGCTCAGGGCCAAGAACAGTTGGAACAGCAGAAT
ATGGGCCAAACAGGATATCTGTGGTAAGCAGTTCCTGCCCCGGCTCAGGG
CCAAGAACAGATGGTCCCCAGATGCGGTCCCGCCCTCAGCAGTTTCTAGA
GAACCATCAGATGTTTCCAGGGTGCCCCAAGGACCTGAAATGACCCTGTG
CCTTATTTGAACTAACCAATCAGTTCGCTTCTCGCTTCTGTTCGCGCGCT
TCTGCTCCCCGAGCTCTATATAAGCAGAGCTCGTTTAGTGAACCGTCAGA
TCGCTAGC

In some embodiments, the vector system comprises at least one retroviral particle, wherein the retroviral particle comprises one or more transduction enhancers as described herein.

In some embodiments, the vector system comprises at least one retroviral particle, wherein the retroviral particle comprises one or more transduction enhancers, wherein the transduction enhancer is selected from the group consisting of a T-cell activation receptor, a NK-cell activation receptor, and a co-stimulatory molecule.

In some embodiments, the one or more transduction enhancers comprise one or more of anti-CD3scFv, CD86, and CD137L.

In some embodiments, at least one polynucleotide sequence is capable of transducing T cells. In some embodiments, at least one polynucleotide sequence is capable of transducing NK cells. In some embodiments, at least one polynucleotide sequence is capable of transducing NKT cells.

In some embodiments, at least one polynucleotide sequence is capable of transducing T cells in vivo. In some embodiments, at least one polynucleotide sequence is capable of transducing NK cells in vivo. In some embodiments, at least one polynucleotide sequence is capable of transducing NKT cells in vivo.

In some embodiments, at least one polynucleotide sequence is capable of transducing T cells in vitro. In some embodiments, at least one polynucleotide sequence is capable of transducing NK cells in vitro. In some embodiments, at least one polynucleotide sequence is capable of transducing NKT cells in vitro.

In some embodiments, the first vector comprises a polynucleotide sequence encoding:
(a) a promoter;
(b) a FK506 binding protein (FKBP) domain or a portion thereof
(c) an IL-2 receptor transmembrane domain
(d) an interleukin-2 receptor subunit gamma (IL2Rγ) domain; and
(e) a first chimeric antigen receptor (CAR).

In some embodiments, the second vector comprises a polynucleotide sequence encoding:
(a) a promoter;
(b) FKBP rapamycin binding (FRB) domain or a portion thereof
(c) an IL-2 receptor transmembrane domain
(d) an interleukin-2 receptor subunit beta (IL2Rβ) domain; and
(e) a second CAR.

In some embodiments, the IL2Rγ domain and IL2Rβ domain heterodimerize. In some embodiments, the IL2Rγ domain and IL2Rβ domain heterodimerize in the presence of a ligand to promote growth and/or survival of a cell. In some embodiments, the IL2Rγ domain and IL2Rβ domain heterodimerize in the presence of rapamycin to promote growth and/or survival of a cell.

In some embodiments, the IL2Rγ domain polypeptide shares at least 75%, at least 80%, at least 85%, at least 90%, at least 95%, at least 99%, or 100% identity to SEQ ID NO: 4.

In some embodiments, the IL2Rγ domain polypeptide shares at least 75%, at least 80%, at least 85%, at least 90%, at least 95%, at least 99%, or 100% identity to SEQ ID NO: 23.

In some embodiments, the IL2Rγ domain polypeptide shares at least 75%, at least 80%, at least 85%, at least 90%, at least 95%, at least 99%, or 100% identity to SEQ ID NO: 24.

In some embodiments, the IL2Rγ domain polypeptide shares at least 75%, at least 80%, at least 85%, at least 90%, at least 95%, at least 99%, or 100% identity to SEQ ID NO: 25.

In some embodiments, the IL2F43 domain polypeptide shares at least 75%, at least 80%, at least 85%, at least 90%, at least 95%, at least 99%, or 100% identity to SEQ ID NO: 5.

In some embodiments, The first CAR may be specific to a cell-surface antigen comprising ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the A1 domain of tenascin-C (TnC A1), thyroglobulin, tumor stromal antigens, vascular endothelial growth factor receptor-2 (VEGFR2), HIV gp120 or a derivate, variant or fragment of these surface antigens.

In some embodiments, The second CAR may be specific to a cell-surface antigen comprising ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the A1 domain of tenascin-C (TnC A1), thyroglobulin, tumor stromal antigens, vascular endothelial growth factor receptor-2 (VEGFR2), HIV gp120 or a derivate, variant or fragment of these surface antigens.

One aspect of the present disclosure provides a method comprising administering to a subject a vector system of any of the embodiments as described in the present disclosure.

### Retroviral Particles

Retroviruses include lentiviruses, gamma-retrovirues, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Retroviral vectors have been generated by multiply attenuating the HIV virulence genes, for example, the genes env, vif, vpr, vpu and nef are deleted, making the vector biologically safe.

Illustrative lentiviral vectors include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

Many retroviral vector systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral vector system into retroviral particles.

As used herein, the terms “retroviral vector” or “lentiviral vector” is intended to mean a nucleic acid that encodes a retroviral or lentiviral cis nucleic acid sequence required for genome packaging and one or more polynucleotide sequence to be delivered into the target cell. Retroviral particles and lentiviral particles generally include an RNA genome (derived from the transfer plasmid), a lipid-bilayer envelope in which the Env protein is embedded, and other accessory proteins including integrase, protease, and matrix protein. As used herein, the terms “retroviral particle” and “lentiviral particle” refers a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a retroviral or lentiviral virion including one or more of the gag structural polypeptides, e.g. p7, p24, and p17, containing a retroviral or lentiviral envelope including one or more of the env encoded glycoproteins, e.g. p41, p120, and p160, containing a genome including one or more retrovirus or lentivirus cis-acting sequences functioning in replication, proviral integration or transcription, containing a genome encoding a retroviral or lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a retroviral or lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of Third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a vector system comprising at least one sequence encoding a receptor that specifically binds to a ligand. In some embodiments, at least one sequence encoding a receptor that specifically binds to the ligand is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments, the retroviral particles comprise transduction enhancers. In some embodiments, the retroviral particles comprise a polynucleotide comprising a sequence encoding a T cell activator protein. In some embodiments, the retroviral particles comprise at least one polynucleotide each comprising a sequence encoding a chimeric antigen receptor. In some embodiments, the retroviral particles comprise tagging proteins.

In some embodiments, the retroviral particles comprise a cell surface receptor that binds to a ligand on a target host cell, allowing host cell transduction. The viral vector may comprise a heterologous viral envelope glycoprotein giving a pseudotyped viral vector. For example, the viral envelope glycoprotein may be derived from RD114 or one of its variants, VSV-G, Gibbon-ape leukaemia virus (GALV), or is the Amphotropic envelope, Measles envelope or baboon retroviral envelope glycoprotein. In some embodiments, the cell-surface receptor is a VSV G protein from the Cocal strain or a functional variant thereof.

Various fusion glycoproteins can be used to pseudotype lentiviral vectors. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

The disclosure further provides various retroviral vectors, including but not limited to gamma-retroviral vectors, alpha-retroviral vectors, and lentiviral vectors.

### Transduction Enhancers

In some embodiments, viral particles according to the present disclosure comprise transduction enhancers.

A “transduction enhancer” as used herein refers to a transmembrane protein that activates T cells. Transduction enhancers may be incorporated into the viral envelopes of viral particles according to the present disclosure. The transduction enhancer may comprise a mitogenic and/or cytokine-based domain. The transduction enhancer may comprise T cell activation receptors, NK cell activation receptors, co-stimulatory molecules, or portions thereof

### Mitogenic Transduction Enhancers

The viral vector of the present invention may comprise a mitogenic transduction enhancer in the viral envelope. In some embodiments, the mitogenic transduction enhancer is derived from the host cell during retroviral vector production. In some embodiments, the mitogenic transduction enhancer is made by the packaging cell and expressed at the cell surface. When the nascent retroviral vector buds from the host cell membrane, the mitogenic transduction enhancer may be incorporated in the viral envelope as part of the packaging cell-derived lipid bilayer.

In some embodiments, the transduction enhancer is host-cell derived. The term “host-cell derived” indicates that the mitogenic transduction enhancer is derived from the host cell as described above and is not produced as a fusion or chimera from one of the viral genes, such as gag, which encodes the main structural proteins; or env, which encodes the envelope protein.

Envelope proteins are formed by two subunits, the transmembrane (TM) that anchors the protein into the lipid membrane and the surface (SU) which binds to the cellular receptors. In some embodiments, the packaging-cell derived mitogenic transduction enhancer of the present invention does not comprise the surface envelope subunit (SU).

The mitogenic transduction enhancer may have the structure: M-S-TM, in which M is a mitogenic domain; S is an optional spacer domain and TM is a transmembrane domain.

### Transduction Enhancer Mitogenic Domains

The mitogenic domain is the part of the mitogenic transduction enhancer which causes T-cell activation. It may bind or otherwise interact, directly or indirectly, with a T cell, leading to T cell activation. In particular, the mitogenic domain may bind a T cell surface antigen, such as CD3, CD28, CD134 and CD137.

CD3 is a T-cell co-receptor. It is a protein complex composed of four distinct chains. In mammals, the complex contains a CD3y chain, a CD35 chain, and two CD3e chains. These chains associate with the T-cell receptor (TCR) and the ζ-chain to generate an activation signal in T lymphocytes. The TCR, ζ-chain, and CD3 molecules together comprise the TCR complex.

In some embodiments, the mitogenic domain may bind to a CD3 ε chain.

CD28 is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). CD134, also known as OX40, is a member of the TNFR-superfamily of receptors which is not constitutively expressed on resting naive T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels.

CD137, also known as 4-1BB, is a member of the tumor necrosis factor (TNF) receptor family. CD137 can be expressed by activated T cells, but to a larger extent on CD8 than on CD4 T cells. In addition, CD137 expression is found on dendritic cells, follicular dendritic cells, natural killer cells, granulocytes and cells of blood vessel walls at sites of inflammation. The best characterized activity of CD137 is its costimulatory activity for activated T cells. Crosslinking of CD137 enhances T cell proliferation, IL-2 secretion survival and cytolytic activity.

The mitogenic domain may comprise all or part of an antibody or other molecule which specifically binds a T-cell surface antigen. The antibody may activate the TCR or CD28. The antibody may bind the TCR, CD3 or CD28. Examples of such antibodies include: OKT3, 15E8 and TGN1412. Other suitable antibodies include:
Anti-CD28: CD28.2, 10F3
Anti-CD3/TCR: UCHT1, YTH12.5, TR66

The mitogenic domain may comprise the binding domain from OKT3, 15E8, TGN1412, CD28.2, 10F3, UCHT1, YTH12.5 or TR66.

The mitogenic domain may comprise all or part of a co-stimulatory molecule such as OX40L and 41 BBL. For example, the mitogenic domain may comprise the binding domain from OX40L or 41 BBL.

### Transduction Enhancer Spacer Domains

The mitogenic transduction enhancer and/or cytokine-based transduction enhancer may comprise a spacer sequence to connect the antigen-binding domain with the transmembrane domain. A flexible spacer allows the antigen-binding domain to orient in different directions to facilitate binding.

The spacer sequence may, for example, comprise an lgG1 Fc region, an lgG1 hinge or a human CD8 stalk or the mouse CD8 stalk. The spacer may alternatively comprise an alternative linker sequence which has similar length and/or domain spacing properties as an lgG1 Fc region, an lgG1 hinge or a CD8 stalk. A human lgG1 spacer may be altered to remove Fc binding motifs.

### Transduction Enhancer Transmembrane Domains

The transmembrane domain is the sequence of the mitogenic transduction enhancer and/or cytokine-based transduction enhancer that spans the membrane. The transmembrane domain may comprise a hydrophobic alpha helix. The transmembrane domain may be derived from CD28. In some embodiments, the transmembrane domain is derived from a human protein.

An alternative option to a transmembrane domain is a membrane-targeting domain such as a GPI anchor. GPI anchoring is a post-translational modification which occurs in the endoplasmic reticulum. Preassembled GPI anchor precursors are transferred to proteins bearing a C-terminal GPI signal sequence. During processing, the GPI anchor replaces the GPI signal sequence and is linked to the target protein via an amide bond. The GPI anchor targets the mature protein to the membrane. In some embodiments, the present tagging protein comprises a GPI signal sequence.

### Cytokine-Based Transduction Enhancers

The viral vector of the present invention may comprise a cytokine-based transduction enhancer in the viral envelope. In some embodiments, the cytokine-based transduction enhancer is derived from the host cell during viral vector production. In some embodiments, the cytokine-based transduction enhancer is made by the host cell and expressed at the cell surface. When the nascent viral vector buds from the host cell membrane, the cytokine-based transduction enhancer may be incorporated in the viral envelope as part of the packaging cell-derived lipid bilayer.

The cytokine-based transduction enhancer may comprise a cytokine domain and a transmembrane domain. It may have the structure C-S-TM, where C is the cytokine domain, S is an optional spacer domain and TM is the transmembrane domain. The spacer domain and transmembrane domains are as defined above.

### Transduction Enhancer Cytokine Domains

The cytokine domain may comprise part or all of a T-cell activating cytokine, such as from IL2, IL7 and IL15. The cytokine domain may comprise part of the cytokine, as long as it retains the capacity to bind its particular receptor and activate T-cells.

IL2 is one of the factors secreted by T cells to regulate the growth and differentiation of T cells and certain B cells. IL2 is a lymphokine that induces the proliferation of responsive T cells. It is secreted as a single glycosylated polypeptide, and cleavage of a signal sequence is required for its activity. Solution NMR suggests that the structure of IL2 comprises a bundle of 4 helices (termed A-D), flanked by 2 shorter helices and several poorly defined loops. Residues in helix A, and in the loop region between helices A and B, are important for receptor binding. The sequence of IL2 is shown as SEQ ID NO: 18.

(SEQ ID NO: 18)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELK GSETTFMCEYADETATIVEFLNRWITFCQSII
STLT

IL7 is a cytokine that serves as a growth factor for early lymphoid cells of both B- and T-cell lineages. The sequence of IL7 is shown as SEQ ID NO: 19.

(SEQ ID NO: 19)
MFHVSFRYIFGLPPLILVLLPVASSDCDIEGKDGKQYESVLMVSIDQLL
DSMKEIGSNCLNNEFNFFKRHICDANKEGMFLFRAARKLRQFLKMNSTG
DFDLHLLKVSEGTTILLNCTGQVKGRKPAALGEAQPTKSLEENKSLKEQ
KKLNDLCFLKRLLQEIKTCWNKILMGTKEH

IL15 is a cytokine with structural similarity to IL2. Like IL2, IL15 binds to and signals through a complex composed of IL2/IL15 receptor beta chain and the common gamma chain. IL15 is secreted by mononuclear phagocytes, and some other cells, following infection by virus(es). This cytokine induces cell proliferation of natural killer cells; cells of the innate immune system whose principal role is to kill virally infected cells. The sequence of IL15 is shown as SEQ ID NO: 20.

(SEQ ID NO: 20)
MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS

The cytokine-based transduction enhancer may comprise one of the following sequences, or a variant thereof:

membrane-IL7:
(SEQ ID NO: 21)
MAHVSFRYIFGLPPLILVLLPVASSDCDIEGKDGKQYESVLMVSIDQLL
DSMKEIGSNCLNNEFNFFKRHICDANKEGMFLFRAARKLRQFLKMNSTG
DFDLHLLKVSEGTTILLNCTGQVKGRKPAALGEAQPTKSLEENKSLKEQ
KKLNDLCFLKRLLQEIKTCWNKILMGTKEHSGGGSPAKPTTTPAPRPPT
PAPTIASQPLSLRPEACRPAAGGAVHTRGLDFACDIYIWAPLAGTCGVL
LLSLVITLYCNHRNRRRVCKCPRPVV
membrane-IL15:
(SEQ ID NO: 22)
MGLVRRGARAGPRMPRGWTALCLLSLLPSGFMAGIHVFILGCFSAGLPK
TEANWVNVISDLKKIEDLIQSMHIDATLYTESDVHPSCKVTAMKCFLLE
LQVISLESGDASIHDTVENLIILANNSLSSNGNVTESGCKECEELEEKN
IKEFLQSFVHIVQMFINTSSPAKPTTTPAPRPPTPAPTIASQPLSLRPE
ACRPAAGGAVHTRGLDFACDIYIWAPLAGTCGVLLLS LVITLYCNHRN
RRRVCKCPRPVV

The cytokine-based transduction enhancer may comprise a variant of the sequence shown as SEQ ID NO: 21 or 22 having at least 70%, 75%, 80%, 85%, 90%, 95%, 98%, or 99% sequence identity, provided that the variant sequence is a cytokine-based transduction enhancer having the required properties i.e. the capacity to activate a T cell when present in the envelope protein of a retroviral or lentiviral vector.

### Illustrative Advantages of Transduction Enhancers

In some embodiments, the present disclosure provides a viral vector with a built-in transduction enhancer. The vector may have the capability to both stimulate the T-cell and to also effect gene insertion. This may produce one or more advantages, including: (1) simplifying the process of T-cell engineering, as only one component needs to be added; (2) avoiding removal of beads and the associated reduction in yield as the virus is labile and does not have to be removed; (3) reducing the cost of T-cell engineering as only one component needs to be manufactured; (4) allowing greater design flexibility, as each T-cell engineering process will involve making a gene transfer vector, the same product can also be made with a transduction enhancer to “fit” the product; (5) shortening the production process: in soluble antigen/bead-based approaches the mitogen and the vector are typically given sequentially separated by one, two or sometimes three days, this can be avoided with the retroviral vector of the present invention since transduction enhancement and viral entry are synchronized and simultaneous; (6) simplifying engineering as there is no need to test a lot of different fusion proteins for expression and functionality; (7) allowing for the possibility to add more than one signal at the same time; and (8) allowing for the regulation of the expression and/or expression levels of each signal/protein separately.

### Illustrative Embodiments of Viral Vectors Comprising Transduction Enhancers

In some embodiments, the viral envelope comprises one or more transduction enhancers. In some embodiments, the transduction enhancers include T cell activation receptors, NK cell activation receptors, and/or co-stimulatory molecules. In some embodiments, one or more transduction enhancers comprise one or more of anti-CD3scFv, CD86, and CD137L. In some embodiments, the transduction enhancers comprise every one of anti-CD3 scFv, CD86, and CD137L.

In some embodiments, the transduction enhancer comprises a mitogenic stimulus, and/or a cytokine stimulus, which is incorporated into a retroviral or lentiviral capsid, such that the virus both activates and transduces T cells. This removes the need to add vector, mitogen and cytokines separately. In some embodiments, the transduction enhancer comprises a mitogenic transmembrane protein and/or a cytokine-based transmembrane protein that is included in the producer or packaging cell, which get(s) incorporated into the retrovirus when it buds from the producer/packaging cell membrane. In some embodiments, the transduction enhancers are expressed as separate cell surface molecules on the producer cell rather than being part of the viral envelope glycoprotein.

In some embodiments, the present disclosure provides a retroviral or lentiviral vector having a viral envelope which comprises:
(i) a mitogenic transduction enhancer which comprises a mitogenic domain and a transmembrane domain; and/or
(ii) a cytokine-based transduction enhancer which comprises a cytokine domain and a transmembrane domain.

In some embodiments, the transduction enhancers are not part of a viral envelope glycoprotein. In some embodiments, the retroviral or lentiviral vector comprises a separate viral envelope glycoprotein, encoded by an env gene. Since the mitogenic stimulus and/or cytokine stimulus are provided on a molecule which is separate from the viral envelope glycoprotein, integrity of the viral envelope glycoprotein is maintained and there is no negative impact on viral titre.

In some embodiments, there is provided a retroviral or lentiviral vector having a viral envelope which comprises:
(i) a viral envelope glycoprotein: and
(ii) a mitogenic transduction enhancer having the structure: M-S-TM in which M is a mitogenic domain; S is an optional spacer and TM is a transmembrane domain; and/or
(iii) a cytokine-based transduction enhancer which comprises a cytokine domain and a transmembrane domain.

In some embodiments, the mitogenic transduction enhancer and/or cytokine-based transduction enhancer are not part of the viral envelope glycoprotein. In some embodiments, they exist as separate proteins in the viral envelope and are encoded by separate genes. In some embodiments, the mitogenic transduction enhancer has the structure:
M-S-TM
   in which M is a mitogenic domain; S is an optional spacer and TM is a transmembrane domain.

In some embodiments, the mitogenic transduction enhancer binds an activating T-cell surface antigen. In some embodiments, the antigen is CD3, CD28, CD134 or CD137. The mitogenic transduction enhancer may comprise an agonist for such an activating T-cell surface antigen.

The mitogenic transduction enhancer may comprise the binding domain from an antibody such as OKT3, 15E8, TGN1412; or a costimulatory molecule such as OX40L or 41 BBL. The viral vector may comprise two or more mitogenic transduction enhancers in the viral envelope. For example, the viral vector may comprise a first mitogenic transduction enhancer which binds CD3 and a second mitogenic transduction enhancer which binds CD28. The cytokine-based transduction enhancer may, for example, comprise a cytokine selected from IL2, IL7 and IL15.

In some embodiments, there is provided a retroviral or lentiviral vector having a viral envelope which comprises:
(a) a first mitogenic transduction enhancer which binds CD3; and
(b) a second mitogenic transduction enhancer which binds CD28.

In some embodiments, there is provided a retroviral or lentiviral vector having a viral envelope which comprises:
(a) a first mitogenic transduction enhancer which binds CD3;
(b) a second mitogenic transduction enhancer which binds CD28; and
(c) a cytokine-based transduction enhancer which comprises IL2.

In some embodiments, there is provided a retroviral or lentiviral vector having a viral envelope which comprises:
(a) a first mitogenic transduction enhancer which binds CD3;
(b) a second mitogenic transduction enhancer which binds CD28;
(c) a cytokine-based transduction enhancer which comprises IL7; and
(d) a cytokine-based transduction enhancer which comprises IL15.

### T Cell Activator Proteins

The present disclosure also provides a viral vector comprising a polynucleotide comprising a sequence encoding a T cell activator protein or T cell activator protein complex. As referred to herein, the terms “T cell activator protein” and “T cell activator protein complex” may be used interchangeably and may refer to a single protein or a complex of separate proteins. In some embodiments, the viral vector transduces a host T cell with the polynucleotide encoding the T cell activator protein such that the T cell expresses said protein. The T cell activator protein may then be engaged to activate the transduced T cell. In some embodiments, the T cell activator protein is a drug-inducible T cell activator protein. In some embodiments, the T cell activator protein forms a chemical-induced signaling complex. In some embodiments, the T cell activator protein forms an engineered complex that initiates a signal into the interior of a cell as a direct outcome of ligand-induced dimerization. The T cell activator protein may be comprised in a homodimer (dimerization of two identical components) or a heterodimer (dimerization of two distinct components). The T cell activator protein complex may be a synthetic complex as described herein. One of skill in the art will recognize that the component parts of the T cell activator protein complex may be composed of a natural or a synthetic component useful for incorporation into the complex. Thus, the examples provided herein are not intended to be limiting. Additional T cell activator proteins that may be implemented herein may be found in WO 2016/139463 and WO 2018/111834, the disclosures of which are incorporated in their entireties herein.

In some embodiments, the T cell activator protein sequence can have a first and a second sequence. The first sequence may encode a first T cell activator protein complex component that can comprise a first extracellular binding domain or portion thereof, a hinge domain, a transmembrane domain, and a signaling domain or portion thereof. The second sequence encodes a second T cell activator protein complex component that can comprise a second extracellular binding domain or a portion thereof, a hinge domain, a transmembrane domain, and a signaling domain or portions thereof. In some embodiments, the first and second components may be positioned such that when expressed, they dimerize in the presence of a ligand.

As used herein, the terms “rapamycin activated cytokine receptor” or “RACR” refer interchangeably to a multipartite receptor that inducibly generates an intracellular signal that promotes proliferation and/or activity of a cell in the presence of rapamycin. The RACR may transduce an IL2-like signal in a T cell in the presence of rapamycin through IL-2R intracellular domain(s) or variants thereof.

In some embodiments, the disclosure provides a protein sequence or sequences for heterodimeric two component T cell activator protein complex. In some embodiments, the first component is an IL2Rγ complex. In some embodiments, the IL2Rγ complex comprises an amino acid sequence as set forth in SEQ ID NO: 4.

(SEQ ID NO: 4)
MPLGLLWLGLALLGALHAQAGVQVETISPGDGRTFPKRGQTCVVHYT
GMLEDGKKFDSSRDRNKPFKFMLGKQEVIRGWEEGVAQMSVGQRAKLTI
SPDYAYGATGHPGIIPPHATLVFDVELLKLGEGSNTSKENPFLFALEAV
VISVGSMGLIISLLCVYFWLERTMPRIPTLKNLEDLVTEYHGNFSAWSG
VSKGLAESLQPDYSERLCLVSEIPPKGGALGEGPGASPCNQHSPYWAPP
CYTLKPET

In some embodiments, the IL2Rγ complex comprises an amino acid sequence as set forth in SEQ ID NO: 23.

(SEQ ID NO: 23)
MPLGLLWLGLALLGALHAQAGVQVETISPGDGRTFPKRGQTCVVHYT
GMLEDGKKFDSSRDRNKPFKFMLGKQEVIRGWEEGVAQMSVGQRAKLTI
SPDYAYGATGHPGIIPPHATLVFDVELLKLGEGSNTSKENPFLFALEAV
VISVGSMGLIISLLCVYFWLERTMPRIPTLKNLEDLVTEYHGNFSAWSG
VSKGLAESLQPDYSERLCLVSEIPPKGGALGEGPGASPCNQHSPYWAPP
CYTLKPET

In some embodiments, the IL2Rγ complex comprises an amino acid sequence as set forth in SEQ ID NO: 24.

(SEQ ID NO: 24)
MPLGLLWLGLALLGALHAQAGVQVETISPGDGRTFPKRGQTCVVHYT
GMLEDGKKFDSSRDRNKPFKFMLGKQEVIRGWEEGVAQMSVGQRAKLTI
SPDYAYGATGHPGIIPPHATLVFDVELLKLGEGSNTSKENPFLFALEAV
VISVGSMGLIISLLCVYFWLERTMPRIPTLKNLEDLVTEYHGNFSAWSG
VSKGLAESLQPDYSERLCLVSEIPPKGGALGEGPGASPCNQHSPYWAPP
CYTLKPET

In some embodiments, the IL2Rγ complex comprises an amino acid sequence as set forth in SEQ ID NO: 25.

(SEQ ID NO: 25)
MPLGLLWLGLALLGALHAQAGVQVETISPGDGRTFPKRGQTCVVHYT
GMLEDGKKFDSSRDRNKPFKFMLGKQEVIRGWEEGVAQMSVGQRAKLTI
SPDYAYGATGHPGIIPPHATLVFDVELLKLGEGSNTSKENPFLFALEAV
VISVGSMGLIISLLCVYFWLERTMPRIPTLKNLEDLVTEYHGNFSAWSG
VSKGLAESLQPDYSERLCLVSEIPPKGGALGEGPGASPCNQHSPYWAPP
CYTLKPET

In some embodiments, the protein sequence for the first T cell activator protein complex component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain.

In some embodiments, the second T cell activator protein complex component is an IL2Rβ complex. In some embodiments, the IL2Rβ complex comprises an amino acid sequence as set forth in SEQ ID NO: 5.

(SEQ ID NO: 5)
MALPVTALLLPLALLLHAARPILWHEMWHEGLEEASRLYFGERNVKG
MFEVLEPLHAMMERGPQTLKETSFNQAYGRDLMEAQEWCRKYMKSGNVK
DLLQAWDLYYHVFRRISKGKDTIPWLGHLLVGLSGAFGFIILVYLLINC
RNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGE
DDAYCTFPSRDDLLLFSPSLLGGPSPPSTAPGGSGAGEERMPPSLQERV
PRDWDPQPLGPPTPGVPDLVDFQPPPELVLREAGEEVPDAGPREGVSFP
WSRPPGQGEFRALNARLPLNTDAYLSLQELQGQDPTHLV

In some embodiments, the IL2Rβ complex comprises an amino acid sequence as set forth in SEQ ID NO: 26.

(SEQ ID NO: 26)
MALPVTALLLPLALLLHAARPILWHEMWHEGLEEASRLYFGERNVKG
MFEVLEPLHAMMERGPQTLKETSFNQAYGRDLMEAQEWCRKYMKSGNVK
DLLQAWDLYYHVFRRISKGKDTIPWLGHLLVGLSGAFGFIILVYLLINC
RNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGE
DDAYCTFPSRDDLLLFSPSLLGGPSPPSTAPGGSGAGEERMPPSLQERV
PRDWDPQPLGPPTPGVPDLVDFQPPPELVLREAGEEVPDAGPREGVSFP
WSRPPGQGEFRALNARLPLNTDAYLSLQELQGQDPTHLV

In some embodiments, the IL2Rβ complex comprises an amino acid sequence as set forth in SEQ ID NO: 27.

(SEQ ID NO: 27)
MALPVTALLLPLALLLHAARPILWHEMWHEGLEEASRLYFGERNVKG
MFEVLEPLHAMMERGPQTLKETSFNQAYGRDLMEAQEWCRKYMKSGNVK
DLLQAWDLYYHVFRRISKGKDTIPWLGHLLVGLSGAFGFIILVYLLINC
RNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGE
DDAYCTFPSRDDLLLFSPSLLGGPSPPSTAPGGSGAGEERMPPSLQERV
PRDWDPQPLGPPTPGVPDLVDFQPPPELVLREAGEEVPDAGPREGVSFP
WSRPPGQGEFRALNARLPLNTDAYLSLQELQGQDPTHLV

In some embodiments, the IL2Rβ complex comprises an amino acid sequence as set forth in SEQ ID NO: 28.

(SEQ ID NO: 28)
MALPVTALLLPLALLLHAARPILWHEMWHEGLEEASRLYFGERNVKG
MFEVLEPLHAMMERGPQTLKETSFNQAYGRDLMEAQEWCRKYMKSGNVK
DLLQAWDLYYHVFRRISKGKDTIPWLGHLLVGLSGAFGFIILVYLLINC
RNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGE
DDAYCTFPSRDDLLLFSPSLLGGPSPPSTAPGGSGAGEERMPPSLQERV
PRDWDPQPLGPPTPGVPDLVDFQPPPELVLREAGEEVPDAGPREGVSFP
WSRPPGQGEFRALNARLPLNTDAYLSLQELQGQDPTHLV

In some embodiments, the second T cell activator protein complex component is an IL7Rα complex. In some embodiments, the IL7Rα complex comprises an amino acid sequence as set forth in SEQ ID NO: 29.

(SEQ ID NO: 29)
MALPVTALLLPLALLLHAARPILWHEMWHEGLEEASRLYFGERNVKG
MFEVLEPLHAMMERGPQTLKETSFNQAYGRDLMEAQEWCRKYMKSGNVK
DLLQAWDLYYHVFRRISKGKDTIPWLGHLLVGLSGAFGFIILVYLLINC
RNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGE
DDAYCTFPSRDDLLLFSPSLLGGPSPPSTAPGGSGAGEERMPPSLQERV
PRDWDPQPLGPPTPGVPDLVDFQPPPELVLREAGEEVPDAGPREGVSFP
WSRPPGQGEFRALNARLPLNTDAYLSLQELQGQDPTHLV

In some embodiments, the protein sequence for the second T cell activator protein complex component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain of the second T cell activator protein complex component.

In some embodiments, the protein sequence may include a linker. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth in SEQ ID NO: 30: GGGS (SEQ ID NO: 30), SEQ ID NO: 31: GGGSGGG (SEQ ID NO: 31), or SEQ ID NO: 32: GGG (SEQ ID NO: 32). Embodiments also comprise a nucleic acid sequence encoding SEQ ID NOs: 30-32. In some embodiments, the transmembrane domain is located N-terminal to the signaling domain, the hinge domain is located N-terminal to the transmembrane domain, the linker is located N-terminal to the hinge domain, and the extracellular binding domain is located N-terminal to the linker.

In some embodiments is provided a protein sequence or sequences for homodimeric two component T cell activator protein complex. In some embodiments, the first T cell activator protein complex component is an IL2Rγ complex. In some embodiments, the IL2Rγ complex comprises an amino acid sequence as set forth in SEQ ID NO: 4.

SEQ ID NO: 4
MPLGLLWLGLALLGALHAQAGVQVETISPGDGRTFPKRGQTCVVHYT
GMLEDGKKFDSSRDRNKPFKFMLGKQEVIRGWEEGVAQMSVGQRAKLTI
SPDYAYGATGHPGIIPPHATLVFDVELLKLGEGSNTSKENPFLFALEAV
VISVGSMGLIISLLCVYFWLERTMPRIPTLKNLEDLVTEYHGNFSAWSG
VSKGLAESLQPDYSERLCLVSEIPPKGGALGEGPGASPCNQHSPYWAPP
CYTLKPET;

In some embodiments, the protein sequence for the first T cell activator protein complex component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain. In some embodiments, the protein sequence of the first T cell activator protein complex component, comprising the first extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NO: 4 or has a sequence identity that is within a range defined by any two of the aforementioned percentages.

In some embodiments, the second T cell activator protein complex component is an IL2Rβ complex or an IL2Rα complex. In some embodiments, the IL2Rβ complex comprises an amino acid sequence as set forth in SEQ ID NO: 5.

(SEQ ID NO: 5)
MALPVTALLLPLALLLHAARPILWHEMWHEGLEEASRLYFGERNVKG
MFEVLEPLHAMMERGPQTLKETSFNQAYGRDLMEAQEWCRKYMKSGNVK
DLLQAWDLYYHVFRRISKGKDTIPWLGHLLVGLSGAFGFIILVYLLINC
RNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGE
DDAYCTFPSRDDLLLFSPSLLGGPSPPSTAPGGSGAGEERMPPSLQERV
PRDWDPQPLGPPTPGVPDLVDFQPPPELVLREAGEEVPDAGPREGVSFP
WSRPPGQGEFRALNARLPLNTDAYLSLQELQGQDPTHLV

In some embodiments, the IL2Rα complex comprises an amino acid sequence as set forth in SEQ ID NO: 33.

(SEQ ID NO: 33)
MALPVTALLLPLALLLHAARPILWHEMWHEGLEEASRLYFGERNVKG
MFEVLEPLHAMMERGPQTLKETSFNQAYGRDLMEAQEWCRKYMKSGNVK
DLLQAWDLYYHVFRRISKGKDTIPWLGHLLVGLSGAFGFIILVYLLINC
RNTGPWLKKVLKCNTPDPSKFFSQLSSEHGGDVQKWLSSPFPSSSFSPG
GLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGE
DDAYCTFPSRDDLLLFSPSLLGGPSPPSTAPGGSGAGEERMPPSLQERV
PRDWDPQPLGPPTPGVPDLVDFQPPPELVLREAGEEVPDAGPREGVSFP
WSRPPGQGEFRALNARLPLNTDAYLSLQELQGQDPTHLV

In some embodiments, the protein sequence for the second T cell activator protein complex component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain of the second T cell activator protein complex component. In some embodiments, the protein sequence of the second T cell activator protein complex component, comprising the second extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NO: 5 or SEQ ID NO: 33, or has a sequence identity that is within a range defined by any two of the aforementioned percentages.

In some embodiments, the sequences for the homodimerizing two component T cell activator protein complex incorporate FKBP F36V domain for homodimerization with the ligand AP1903.

In some embodiments, the at least one T-cell activator protein comprises a first receptor protein comprising a first dimerization domain and a second receptor protein comprising a second dimerization domain, wherein the first dimerization domain and the second dimerization domain specifically bind to one another in response to a molecule. The molecule bound by the T cell activator protein, alternatively termed the term “ligand” or “agent”, refers to a molecule that has a desired biological effect. In some embodiments, a ligand is recognized by and bound by an extracellular binding domain, forming a tripartite complex comprising the ligand and two binding T cell activator protein complex components. Ligands include, but are not limited to, proteinaceous molecules, comprising, but not limited to, peptides, polypeptides, proteins, post-translationally modified proteins, antibodies etc.; small molecules (less than 1000 daltons), inorganic or organic compounds; and nucleic acid molecules comprising, but not limited to, double-stranded or single-stranded DNA, or double-stranded or single-stranded RNA (e.g., antisense, RNAi, etc.), aptamers, as well as triple helix nucleic acid molecules. Ligands can be derived or obtained from any known organism (comprising, but not limited to, animals (e.g., mammals (human and non-human mammals)), plants, bacteria, fungi, and protista, or viruses) or from a library of synthetic molecules. In some embodiments, the ligand is a protein, an antibody, a small molecule, or a drug. In some embodiments, the ligand is rapamycin or a rapamycin analog (rapalogs). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Thus, in some embodiments, the rapalog is everolimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophernolic acid, benidipine hydrochloride, rapamine, AP23573, or AP1903, or metabolites, derivatives, and/or combinations thereof. In some embodiments, the ligand is an IMID-class drug (e.g. thalidomide, pomalidimide, lenalidomide or related analogues).

In some embodiments, the molecule is selected from FK1012, tacrolimus (FK506), FKCsA, rapamycin, coumermycin, gibberellin, HaXS, TMP-HTag, and ABT-737 or functional derivatives thereof.

### Chimeric Antigen Receptor

The terms “Chimeric antigen receptor” or “CAR” or “Chimeric T cell receptor” refer to a synthetically designed receptor comprising a ligand binding domain of an antibody or other protein sequence that binds to a molecule, a transmembrane domain, one or more intracellular signaling domains, and one or more co-stimulatory domains. The ligand binding domain is linked via a spacer domain to one or more intracellular signaling domains of a T cell or other receptors, such as a costimulatory domain. Chimeric receptors can also be referred to as artificial T cell receptors, chimeric T cell receptors, chimeric immunoreceptors, and chimeric antigen receptors (CARs). These CARS are engineered receptors that can graft an arbitrary specificity onto an immune receptor cell. In some embodiments, the spacer for the chimeric antigen receptor is selected (e.g., for a particular length of amino acids in the spacer) to achieve desired binding characteristics for the CAR. CARS having varying lengths of spacers, e.g., presented on cells are then screened for the ability to bind or interact with a molecule to which the CAR is directed.

In some embodiments herein, the CAR comprises one or more intracellular signaling domains. In some embodiments, the intracellular signaling domain is derived from CD27, CD28, 4-IBB, OX40, CD30, CD40, ICOS, lymphocyte function-associated antigen-I (LFA-1), CD2, CD7, LIGHT, NKG2C, B7-H3, or a ligand that specifically binds with CD83, or a portion thereof.

In some embodiments, the CAR comprises one or more co-stimulatory domains. A “co-stimulatory domain” refers to a signaling moiety that provides to T cells a signal which, in addition to the primary signal provided by for instance the CD3 zeta chain of the TCR/CD3 complex, mediates a T cell response, including, but not limited to, activation, proliferation, differentiation, cytokine secretion, and the like. A co-stimulatory domain can include all or a portion of, but is not limited to, CD27, CD28, 4-IBB, OX40, CD30, CD40, ICOS, lymphocyte function-associated antigen-I (LFA-1), CD2, CD7, LIGHT, NKG2C, B7-H3, or a ligand that specifically binds with CD83. In some embodiments, the co-stimulatory domain is an intracellular signaling domain that interacts with other intracellular mediators to mediate a cell response including activation, proliferation, differentiation and cytokine secretion, and the like. In some embodiments, herein the co-stimulatory domain comprises 41bb and CD3zeta. In some embodiments, the vector system comprises a CAR specific for CD19. In some embodiments, the vector system comprises a CAR specific for CD20. In some embodiments, the T cell further comprises an 806 CAR (anti-EGFR 806-41BB-CD3zeta CAR).

In some embodiments, the CAR is a dimerization activated receptor initiation complex (DARIC). A DARIC provides a binding component and a signaling component that are each expressed as separate fusion proteins but contain an extracellular multimerization mechanism (bridging factor) for recoupling of the two functional components on a cell surface (see U.S. Pat. Appl. No. 2016/0311901, hereby expressly incorporated by reference in its entirety). Importantly, the bridging factor in the DARIC system forms a heterodimeric receptor complex, which does not produce significant signaling on its own. The described DARIC complexes only initiate physiologically relevant signals following further co-localization with other DARIC complexes. Thus, they do not allow for the selective expansion of desired cell types without a mechanism for further multimerization of DARIC complexes (such as by e.g., contact with a tumor cell that expresses a ligand bound by a binding domain incorporated into one of the DARIC components).

In some embodiments, the antigen-binding portion of a CAR may comprise an antigen-binding portion of an antibody or an antigen-binding antibody derivative. An antigen-binding portion or derivative of an antibody may be a Fab, Fab′, F(ab′)2, Fd, Fv, scFv, a diabody, a linear antibody, a single-chain antibody, a minibody, or the like. In some embodiments, the antigen-binding portion of a CAR may comprise a DARPin or centyrin.

The CAR may bind to a molecule associated with a disease or disorder. In some embodiments, the antigen to which the CARS bind or interact can be presented on a substrate, such as a membrane, bead, or support (e.g., a well) or a binding agent, such as a lipid (e.g., PLE), hapten, ligand, or antibody, or binding fragment thereof. In some embodiments, the CAR has specificity for an antigen present on a cancer cell. In some embodiments, the CAR has specificity for a pathogen, such as a virus or bacterium. By one approach, the substrate comprising the desired antigen is contacted with a plurality of cells comprising a CAR specific for said antigen and the level or amount of binding of the cells comprising the CAR to the antigen present on the substrate or binding agent is determined. Such an evaluation of binding may include staining for cells bound to adaptor molecules or evaluation of fluorescence or loss of fluorescence. Again, modifications to the CAR structure, such as varying spacer lengths, can be evaluated in this manner. In some approaches, a target cell is also provided such that the method comprises contacting a cell, such as a T cell, which comprises a CAR that is specific for an adaptor molecule comprising a target moiety and an antigen, in the presence of a target cell, such as a cancer cell or bacterial cell, or a target virus and evaluating the binding of the cell comprising the CAR to the adaptor molecule and/or evaluating the binding of the cell comprising the CAR to the target cell or target virus. The variation of the different elements of the CAR can, for example, lead to stronger binding affinity for a specific epitope or antigen.

In some embodiments described herein, the CAR is specific for a lipid or peptide that targets a tumor or cancer cell, wherein the lipid or peptide comprises an antigen and the CAR can specifically bind to said lipid through an interaction with said antigen. In some embodiments, the lipid is a phospholipid ether. In some embodiments described herein, the CAR is specific for a phospholipid ether, wherein the phospholipid ether comprises an antigen and the CAR specifically binds to said phospholipid ether through an interaction with said antigen.

In some embodiments, the CAR is specific for an antigen affixed to an antibody or binding fragment thereof, wherein the CAR specifically binds to said antibody or binding fragment thereof through an interaction with said antigen. Exemplary antigens which can be conjugated to said antibody or binding fragment thereof include a poly(his) tag, Strep-tag, FLAG-tag, VS-tag, Myc-tag, HA-tag, NE-tag, biotin, digoxigenin, dinitrophenol, green fluorescent protein (GFP), yellow fluorescent protein, orange fluorescent protein, red fluorescent protein, far red fluorescent protein, or fluorescein (e.g., fluorescein isothiocyanate (FITC)). In some embodiments, the antibody or binding fragment thereof is specific for an antigen or ligand present on a cancer cell or a pathogen (e.g., viral or bacterial pathogen). In some embodiments, the antibody or binding fragment thereof is specific for an antigen or ligand present on a tumor cell, a virus, preferably a chronic virus (e.g., a hepatitis virus, such as HBV or HCV, or HIV), or a bacterial cell.

In some embodiments, the CAR nucleic acid comprises a polynucleotide coding for a transmembrane domain. The transmembrane domain provides for anchoring of the chimeric receptor in the membrane.

In some embodiments, a complex is provided, wherein the complex comprises a CAR joined to a lipid wherein the lipid comprises an antigen and the CAR is joined to said lipid through an interaction with said antigen.

In some embodiments, a complex is provided, wherein the complex comprises a CAR joined to an antibody or binding fragment thereof, wherein the antibody or binding fragment thereof comprises an antigen (e.g., a poly(his) tag, Strep-tag, FLAG-tag, VS-tag, Myc-tag, HA-tag, NE-tag, biotin-digoxigenin, dinitrophenol, green fluorescent protein (GFP), yellow fluorescent protein, orange fluorescent protein, red fluorescent protein, far red fluorescent protein, or fluorescein (e.g., fluorescein isothiocyanate (FITC)) and the CAR is joined to said antibody or binding fragment thereof through an interaction with said antigen. In some embodiments, the antibody or binding fragment thereof is further joined to an antigen or ligand present on a cancer cell or a pathogen (e.g., viral or bacterial pathogen). In some embodiments, the antibody or binding fragment thereof is joined to an antigen or ligand present on a tumor cell, a virus, preferably a chronic virus (e.g., a hepatitis virus, such as HBV or HCV, or HIV), or a bacterial cell. In some embodiments, the antigen is present on an antibody or binding fragment thereof, which are specific for an antigen on a cancer cell or pathogen (e.g., a virus or bacterial cell), and said antigen is bound by a CAR present on the surface of a cell (e.g., a T cell) such that the cell having the CAR is redirected to the cancer cell or pathogen.

In some embodiments, the CAR or T cell activator protein of the present disclosure confers resistance to an immunosuppressive or anti-proliferative agent to the immune cell. In some cases, the lentiviral vector facilitates selective expansion of target cells by conferring resistance to an immunosuppressive or anti-proliferative agent to transduced cells, facilitating selective expansion of target cells. The present disclosure provides lentiviral vectors that comprise any of the nucleic sequences that confer resistance to an immunosuppressive or anti-proliferative agent. Examples of immunosuppressive or anti-proliferative agents include, without limitation, rapamycin or a derivative thereof, a rapalog or a derivative thereof, tacrolimus or a derivative thereof, cyclosporine or a derivative thereof, methotrexate or derivatives thereof, and mycophenolate mofetil (MMF) or derivatives thereof. Various resistance genes are known in the art. Resistance to rapamycin may be conferred by a polynucleotide sequence encoding the protein domain FRB, found in the mTOR domain and known to be the target of the FKBP-rapamycin complex. Resistance to tacrolimus may be conferred by a polynucleotide sequence encoding the calcineurin mutant CNa22 or calcineurin mutant CNb30. Resistance to cyclosporine may be conferred by a polynucleotide sequence encoding the calcineurin mutant CNa12 or calcineurin mutant CNb30. These calcineurin mutants are described in Brewin et al. (2009) Blood 114:4792-803. Resistance to methotrexate can be provided by various mutant forms of di-hydrofolate reducatse (DHFR), Volpato et al. (2011) J Mol Recognition 24:188-198, and resistance to MMF can be provided by various mutant forms of inosine monophosphate dehydrogenase (IMPDH), Yam et al. (2006) Mol Ther 14:236-244.

In some embodiments, the chimeric antigen receptor comprises an antigen binding molecule that specifically binds to a target antigen. In some embodiments, the target antigen is CD3, CD28, CD134 and CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the A1 domain of tenascin-C (TnC A1), thyroglobulin, tumor stromal antigens, vascular endothelial growth factor receptor-2 (VEGFR2), HIV gp120 or a derivate, variant or fragment of these surface antigens.

Immunosuppressive or anti-proliferative agents (e.g., immunosuppressive drugs) are commonly used prior to, during, and/or after ACT. In some cases, use of an immunosuppressive drug may improve treatment outcomes. In some cases, use of an immunosuppressive drug may diminish side effects of treatment, such as, without limitation, acute graft-versus-host disease, chronic graft-versus-host disease, and post-transplant lymphoproliferative disease. The present disclosure contemplates use of immunosuppressive drugs with any of the methods of treating or preventing a disease or condition of the present disclosure, including, without limitation, methods of the present disclosure in which the lentiviral vector confers resistance to an immunosuppressive drug to transduced cells.

### Polynucleotides

The present disclosure also relates to nucleic acids and polynucleotides encoding the disclosed transduction enhancers, T cell activator proteins, adaptor molecules, and CARs. The nucleic acid may be in the form of a construct comprising a plurality of sequences encoding any of the aforementioned proteins. As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence. The nucleic acid may produce a polypeptide which comprises one or more sequences encoding a mitogenic transduction enhancer and/or one or more sequences encoding a cytokine-based transduction enhancer. The cleavage site may be self-cleaving, such that when the polypeptide is produced, it is immediately cleaved into the receptor component and the signaling component without the need for any external cleavage activity.

Various self-cleaving sites are known, including the Foot-and-Mouth disease virus (FMDV) 2a self-cleaving peptide and various variants and 2A-like peptides.

The co-expressing sequence may be an internal ribosome entry sequence (IRES). The co-expressing sequence may be an internal promoter.

In some embodiments, the polynucleotide encodes a protein that confers resistance to an antiangiogenic agent to the immune cell transduced with it.

### Viral Particle Tagging Proteins

The viral envelope of the viral vector may also comprise a tagging protein which comprises a binding domain which binds to a capture moiety and a transmembrane domain.

The tagging protein may comprise: a binding domain which binds to a capture moiety; a spacer; and a transmembrane domain.

The tagging protein facilitates purification of the viral vector from cellular supernatant via binding of the tagging protein to the capture moiety. ‘Binding domain’ refers to an entity, for example an epitope, which is capable recognizing and specifically binding to a target entity, for example a capture moiety. The binding domain may comprise one or more epitopes which are capable of specifically binding to a capture moiety. For example the binding domains may comprise at least one, two, three, four or five epitopes capable of specifically binding to a capture moiety. Where the binding domain comprises more than one epitope, each epitope may be separated by a linker sequence, as described herein.

The binding domain may be releasable from the capture moiety upon the addition of an entity which has a higher binding affinity for the capture moiety compared to the binding domain.

The binding domain may comprise one or more streptavidin-binding epitope(s). For example, the binding domain may comprise at least one, two, three, four or five streptavidin-binding epitopes.

Streptavidin is a 52.8 kDa protein purified from the bacterium Streptomyces avidinii. Streptavidin homo-tetramers have a very high affinity for biotin (vitamin B7 or vitamin H). Streptavidin is well known in the art and is used extensively in molecular biology and bio-nanotechnology due to the streptavidin-biotin complex's resistance to organic solvents, denaturants, proteolytic enzymes, and extremes of temperature and pH. The strong streptavidin-biotin bond can be used to attach various biomolecules to one another or on to a solid support. Harsh conditions are needed to break the streptavidin-biotin interaction, however, which may denature a protein of interest being purified.

The binding domain may be, for example, a biotin mimic. A ‘biotin mimic’ refers to a short peptide sequence—for example 6 to 20, 6 to 18, 8 to 18 or 8 to 15 amino acids—which specifically binds to streptavidin. As described above, the affinity of the biotin/streptavidin interaction is very high. It is therefore an advantage of the present invention that the binding domain may comprise a biotin mimic which has a lower affinity for streptavidin compared to biotin itself.

In particular, the biotin mimic may bind streptavidin with a lower binding affinity than biotin, so that biotin may be used to elute streptavidin-captured retroviral vectors. For example, the biotin mimic may bind streptavidin with a Kd of 1 nM to 100 uM.

The biotin mimic may be selected from the following group: Strep-tag II, Flankedccstreptag and ccstreptag. The binding domain may comprise more than one biotin mimic. For example the binding domain may comprise at least one, two, three, four or five biotin mimics. Where the binding domain comprises more than one biotin mimic, each mimic may be the same or a different mimic.

The present disclosure also provides viral particles that may be purified and methods of purification of the same. In some embodiments, the viral envelope of the viral vector may also comprise a tagging protein which comprises: a binding domain which binds to a capture moiety; a spacer; and a transmembrane domain, which tagging protein facilitates purification of the viral vector from cellular supernatant via binding of the tagging protein to the capture moiety.

The binding domain of the tagging protein may comprise one or more streptavidin-binding epitope(s). The streptavidin-binding epitope(s) may be a biotin mimic, such as a biotin mimic which binds streptavidin with a lower affinity than biotin, so that biotin may be used to elute streptavidin-captured retroviral vectors produced by the packaging cell. Examples of suitable biotin mimics include: Strep-tag II, Flankedccstretag, and ccstreptag. The viral vector of the first aspect of the invention may comprise a nucleic acid sequence encoding a T-cell receptor or a chimeric antigen receptor. The viral vector may be a virus-like particle (VLP).

### Production/Packaging Cell Lines

The present disclosure provides a host cell for the production of viral particles according to the disclosure. In some embodiments, the host cell expresses a mitogenic transduction enhancer and/or a cytokine-based transduction enhancer at the cell surface. The host cell may be for the production of viral vectors according to the foregoing embodiments. In some embodiments, the host cell may comprise tagging proteins useful for the purification of the viral particles.

The host cell may be a packaging cell and comprise one or more of the following genes: gag, pol, env and rev. A packaging cell for a retroviral vector may comprise gag, pol and env genes. A packaging cell for a lentiviral vector may comprises gag, pol, env and rev genes.

The host cell may be a producer cell and comprise gag, pol, env and optionally rev genes and a retroviral or lentiviral vector genome. In a typical recombinant retroviral or lentiviral vector for use in gene therapy, at least part of one or more of the gag-pol and env protein coding regions may be removed from the virus and provided by the packaging cell. This makes the viral vector replication-defective as the virus is capable of integrating its genome into a host genome but the modified viral genome is unable to propagate itself due to a lack of structural proteins.

Packaging cells are used to propagate and isolate quantities of viral vectors i.e. to prepare suitable titres of the retroviral vector for transduction of a target cell.

In some instances, propagation and isolation may entail isolation of the retroviral gagpol and env (and in the case of lentivirus, rev) genes and their separate introduction into a host cell to produce a packaging cell line. The packaging cell line produces the proteins required for packaging retroviral DNA but it cannot bring about encapsidation due to the lack of a psi region. However, when a recombinant vector carrying a psi region is introduced into the packaging cell line, the helper proteins can package the psi-positive recombinant vector to produce the recombinant virus stock.

A summary of the available packaging lines is presented in Coffin, J. M., et al. (1997) Retroviruses 449.

Packaging cells have also been developed in which the gag, pol and env (and, in the case of lentiviral vectors, rev) viral coding regions are carried on separate expression plasmids that are independently transfected into a packaging cell line, so that three recombinant events are required for wild type viral production.

Transient transfection avoids the longer time required to generate stable vector-producing cell lines and is used if the vector or retroviral packaging components are toxic to cells. Components typically used to generate retroviral/lentivial vectors include a plasmid encoding the Gag/Pol proteins, a plasmid encoding the Env protein (and, in the case of lentiviral vectors, the rev protein), and the retroviral/lentiviral vector genome. Vector production involves transient transfection of one or more of these components into cells containing the other required components. The packaging cells of the present invention may be any mammalian cell type capable of producing retroviral/lentiviral vector particles. The packaging cells may be 293T-cells, or variants of 293T-cells which have been adapted to grow in suspension and grow without serum.

The packaging cells may be made by transient transfection with
a) the transfer vector
b) a gagpol expression vector
c) an env expression vector. The env gene may be a heterologous, resulting in a pseudotyped retroviral vector. For example, the env gene may be from RD1 14 or one of its variants, VSV-G, the Gibbon-ape leukaemia virus (GALV), the Amphotropic envelope or Measles envelope or baboon retroviral envelope glycoprotein.

In the case of lentiviral vector, transient transfection with a rev vector is also performed.

The present disclosure provides host cells expressing viral particles according to the foregoing embodiments. In some embodiments, the host cells express, at the cell surface, one or more transduction enhancers. In some embodiments, the present invention provides a host cell which expresses, at the cell surface,
(a) a mitogenic transduction enhancer comprising a mitogenic domain and a transmembrane domain; and/or
(b) a cytokine-based transduction enhancer which comprises a cytokine domain and a transmembrane domain;
such that a retroviral or lentiviral vector produced by the packaging cell is as described in the foregoing embodiments.

In some embodiments, the host cell may also express, at the cell surface, a tagging protein which comprises: a binding domain which binds to a capture moiety; and a transmembrane domain, which tagging protein facilitates purification of the viral vector from cellular supernatant via binding of the tagging protein to the capture moiety, such that a retroviral or lentiviral vector produced by the packaging cell has the characteristics describing in the foregoing sections.

The tagging protein may also comprise a spacer between the binding domain and the transmembrane domain.

The term host cell may be used to describe a packaging cell or a producer cell. A packaging cell may comprise one or more of the following genes: gag, pol, env and/or rev. A producer cell may comprise gag, pol, env and optionally rev genes and also comprises a retroviral or lentiviral genome. In some embodiments, the host cell may be any suitable cell line stably expressing mitogenic and/or cytokine transduction enhancers. It may be transiently transfected with transfer vector, gagpol, env (and rev in the case of a lentivirus) to produce replication incompetent retroviral/lentiviral vector.

The present disclosure also provides a method for making a host cell according to the above, which comprises the step of transducing or transfecting a cell with a nucleic acid encoding one or more transduction enhancers. Also provided is a method for producing a viral vector according to the foregoing embodiments which comprises the step of expressing a retroviral or lentiviral genome in a cell according to the second aspect of the invention.

### Transgenic Immune Cells

The present disclosure provides a method for making an activated transgenic immune cell, which comprises the step of contacting an immune cell with a viral vector according to any of the foregoing embodiments. The immune cells may be transduced in vivo or ex vivo. In some embodiments, the viral vectors are administered to a living subject such that the immune cells are transduced in vivo without any need to isolate and manipulate host cells ex vivo. In some embodiments, immune cells are manipulated ex vivo and then returned to the subject in need thereof.

The immune cells generally are mammalian cells, and typically are human cells, more typically primary human cells, e.g., allogeneic or autologous donor cells. The cells may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immune systems, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, herein, the cells provided are cytotoxic T lymphocytes. A “Cytotoxic T lymphocyte” (CTL) may include but is not limited to, for example, a T lymphocyte that expresses CD8 on the surface thereof (e.g., a CD8+ T cell). In some embodiments, such cells are preferably “memory” T cells (TM cells) that are antigen-experienced. In some embodiments, the cell is a precursor T cell. In some embodiments, the precursor T cell is a hematopoietic stem cell. In some embodiments, the cell is a CD8+ T cytotoxic lymphocyte cell selected from the group consisting of naive CD8+ T cells, central memory CD8+ T cells, effector memory CD8+ T cells and bulk CD8+ T cells. In some embodiments, the cell is a CD4+ T helper lymphocyte cell that is selected from the group consisting of naive CD4+ T cells, central memory CD4+ T cells, effector memory CD4+ T cells, and bulk CD4+ T cells.

Suitable populations of engineered cells that may be used in the methods include, but are not limited to, any immune cells with cytolytic activity, such as T cells. Illustrative sub-populations of T cells include, but are not limited to, those expressing CD3+ including CD3+CD8+ T cells, CD3+CD4+ T cells, and NKT cells.

The cells used in the vector system of the present disclosure are cytotoxic lymphocytes selected from cytotoxic T cells (also variously known as cytotoxic T lymphocytes, CTLs, T killer cells, cytolytic T cells, CD8+ T cells, and killer T cells), natural killer (NK) cells, and lymphokine-activated killer (LAK) cells. Upon activation, each of these cytotoxic lymphocytes triggers the destruction of target tumor cells.

“Natural Killer” NK cells are a cytotoxic lymphocyte that represents a major component of the innate immune system. NK cells respond to tumor formation and cells infected by viruses and induce apoptosis (cell death) in infected cells.

The NK cells used in the vector system transduction of the present disclosure may comprise the NK cells as described in literature as well as NK cells which express one or more markers from any source.

In some embodiments, the NK cells are defined as CD3− CD56+ cells.

In some embodiments, the NK cells are defined as CD7+ CD127− NKp46+T-bet+ Eomes+ cells.

In some embodiments, the NK cells are defined as CD3− CD56dim CD16+ cells.

In some embodiments, the NK cells are defined as CD3− CD56bright CD16− cells.

In some embodiments, the NK cells comprise cell surface receptors that include, but are not limited to, human killer immunoglobulin-like receptors (KIRs), mouse Ly49 family receptors, CD94-NKG2 heterodimeric receptors, NKG2D, natural cytotoxicity receptors (NCRs), or any combination thereof.

In some embodiments, the T cells or NK cells are allogeneic donor cells.

In some embodiments, the T cells or NK cells are autologous donor cells.

As used herein, any reference to a transgenic T cell or transduced T cell, or the use thereof, may also be applied to any of the other immune cell types disclosed herein.

The present disclosure also provides transgenic immune cells comprising one or more exogenous nucleic acid molecules. In some embodiments, the transgenic immune cells comprise at least two polynucleotides encoding the vector system of the present disclosure. In some embodiments, the transgenic immune cells comprise polynucleotides encoding transduction enhancers. In some embodiments, the transgenic immune cells comprise polynucleotides encoding T cell activator proteins. In some embodiments, the transgenic immune cells comprise at least two polynucleotides encoding the vector system of the present disclosure and polynucleotides encoding T cell activator proteins.

Methods of Treating Subjects with the Disclosed Compositions

The present disclosure provides methods of treating a subject in need thereof with the compositions, therapeutic compositions, cells, vectors, and polynucleotides disclosed herein. In some embodiments, the disclosure provides a method of treating cancer and/or killing cancer cells in a subject, comprising administering a therapeutically effective amount of the disclosed viral particles to the subject.

In some embodiments, a method disclosed herein may be used to treat cancer and/or kill cancer cells in a subject by administering a therapeutically effective amount of the lentiviral particles according to any of the foregoing embodiments. In some embodiments, a method disclosed herein may be used to treat cancer and/or kill cancer cells by administering a vector system.

The present disclosure also provides a method of treating cancer and/or killing cancer cells in a subject, comprising administering the system of any of the foregoing embodiments to the subject.

### Modes of Administration and Pharmaceutical Compositions

The disclosed viral particles may be administered in a number of ways depending upon whether local or systemic treatment is desired.

The compositions or embodiments described herein may be formulated for administration in a pharmaceutical carrier in accordance with known techniques. See, e.g., Remington, The Science and Practice of Pharmacy (21st Ed. 2005). In the manufacture of a pharmaceutical formulation, the composition is typically admixed with, inter alia, an acceptable carrier. The carrier must, of course, be acceptable in the sense of being compatible with any other ingredients in the formulation and must not be deleterious to the subject. The carrier may be a solid or a liquid, or both, and is preferably formulated with the compound as a unit-dose formulation, for example, a tablet, which may contain from 0.01% or 0.5% to 95% or 99% by weight of the active compound. One or more embodiments may be incorporated in the formulations disclosed herein, which may be prepared by any of the well-known techniques of pharmacy comprising admixing the components, optionally including one or more accessory ingredients.

Furthermore, a “pharmaceutically acceptable” component such as a sugar, carrier, excipient or diluent of a composition according to the present disclosure is a component that (i) is compatible with the other ingredients of the composition in that it can be combined with the compositions of the present disclosure without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as saline solutions, water, emulsions such as oil/water emulsion, microemulsions and various types of wetting agents.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intraarterial, intrathecal, intraventricular, intraurethral, intracranial, intratumoral, intrasynovial injection or infusions; and kidney dialytic infusion techniques. In a preferred embodiment, parenteral administration of the compositions of the present disclosure comprises intravenous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present compositions of viral particles may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In the context of administering viral particles, the amount of viral particles and time of administration of such particles will be within the purview of the skilled artisan having benefit of the present teachings. In some embodiments, the administration of therapeutically-effective amounts of the disclosed compositions may be achieved by a single administration, such as for example, a single injection of sufficient numbers of viral particles to provide therapeutic benefit to the patient undergoing such treatment. In some embodiments, the subject is provided multiple, or successive administrations of the lentiviral vector compositions, either over a relatively short, or a relatively prolonged period of time, as may be determined by the medical practitioner overseeing the administration of such compositions. For example, the number of infectious particles administered to a mammal may be on the order of about 10⁷, 10⁸, 10⁹, 10¹⁰, 10¹¹, 10¹², 10¹³, or even higher, viral particles/ml given either as a single dose, or divided into two or more administrations as may be required to achieve therapy of the particular disease or disorder being treated. In some embodiments, a subject may be administered two or more different viral vector compositions, either alone, or in combination with one or more other therapeutic drugs to achieve the desired effects of a particular therapy regimen. In some embodiments, the viral vectors are administered in combination with the transgenic immune cells. In some embodiments, the viral vectors are administered in combination with immune cells that have not yet been transduced. The phrase “in combination” may comprise at the same time or at different times within a short period of time, e.g., within one week, one day, twelve hours, six hours, one hour, thirty minutes, ten minutes, five minutes, or one minute.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the invention.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what is regarding as the invention.

### Example 1: Lentiviral Particle Production

Four T175 flasks were seeded with 27×10⁶ HEK293T cells in 5% DMEM media. Transfection mixes were prepared according to Table 2 by the addition of plasmids according to Table 1 to SF media (DMEM without additives), followed by the addition of polyethylenimine (PEI) to the mixture, mixing by vortex and incubating at room temperature (RT) for 20 minutes. The transfection mixture was then added to 25 ml fresh 5% DMEM per T175 flask (100 ml total). Seeding media was then aspirated from the 293T cells and transfection media was added. After two days of incubation, the supernatant was harvested and 25 ml was added back to the cells. The next day, the supernatant was harvested, filtered through a 0.45 micron filter, centrifuged at 25,400 rpm for 105 minutes at 4° C., and resuspended in 450 μl PBS.

TABLE 1

| | | | | Total ul |
|---|---|---|---|---|
| | Genscript | Umoja | concen- | needed for |
| plasmid | Lot | ID | tration | 4x T175 |
| pRRL-MND-Human- | U005HFD240-12 | | 1 ug/ul | 112 |
| Frb-RACCRb- | | | | |
| CD19_CAR-VTw | | | | |
| pRRL-MND-human- | U005HFD240-16 | | 1 ug/ul | 112 |
| Frb-RACCRg- | | | | |
| CD20_CAR-VTw | | | | |
| pUM-MDLg-pRRE | U9159FE040-1 | 89 | 1 ug/ul | 56 |
| pUM-RSV-REV | U9159FE040-2 | 85 | 1 ug/ul | 56 |
| p57M-MND-Cocal- | U9159FE040-2 | 33 | 1 ug/ul | 56 |
| wPRE-BGHpolyA | | | | |

TABLE 2

| | |
|---|---|
| CF10 | 4x T175 |

| | | |
|---|---|---|
| sq cm | 6320 | 700 |

| | | | |
|---|---|---|---|
| transfer 1 | 1000 | ug | 112 |
| transfer 2 | 1000 | ug | 112 |
| reqpol | 500 | ug | 56 |
| rev | 500 | ug | 56 |
| env | 500 | ug | 56 |
| sf media | 90 | ml | 10 ml |
| PEI | 7.5 | ml | 1176 |

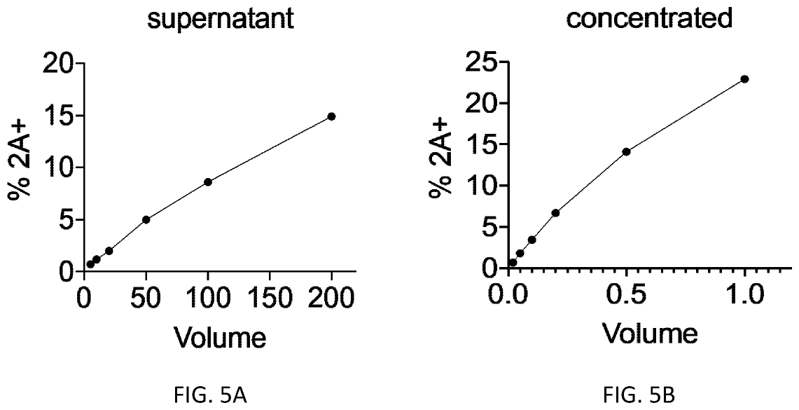

For the lentiviral particle titer determination, 293T cells were seeded at a concentration of 1×10⁵ cells/well in 12 well plates. The next day, cells were counted and transduced using the mixture as described. Supernatant volumes analyzed for the % of 2A self-processing peptide include: 200 μl, 100 μl, 50 μl, 20 μl, 10 μl, and 5 μl as shown in FIG. 5A. Concentrated supernatant volumes analyzed for the % of 2A peptide include: 1 μl, 0.5 μl, 0.2 μl, 0.1 μl, 0.05 μl, and 0.02 μl as shown in FIG. 5B.

Three days after the lentiviral particle titer transduction, the cells were stained for 30 minutes with each of CD20-His, His-PE, CD19-FITC, and 2A. The cells were then analyzed by flow cytometry to measure the lentiviral titer produced. In the supernatant samples, the lentviral titer was 3.65×10⁵ TU/ml (FIG. 5A), and in the concentrated samples, the lentviral titer was 1.12×10⁸ TU/ml (FIG. 5B).

### Example 2: Dual Vector System Cell Transduction

This example demonstrates expression of a CD19 and CD20 split RACR system in primary human T cells.

On Day 1 of the protocol, primary CD3+ T-cells (˜15 million cells, Bloodworks donor 3251BW) were thawed and placed in RPMI-1640 media comprising 10% FBS, Penicillin, Streptomycin, and 50 IU/ml huIL2 (hereinafter “RPMI complete”).

On Day 2, the T cells were bead stimulated (1:1) with anti-CD3 anti-CD28 Thermofisher Dynabeads.

On Day 4, the bead activated T cells were transduced with 12.5 multiplicity of infection (MOI) of the lentiviral preparation as described above. An aliquot of untransduced T cells (MOI 0) were left as a control.

On Day 6, the transduced T cells were divided as needed to maintain approximately 0.5×10⁶ cells/ml in RPMI with stimulated conditions.

On Day 7, the cells were diluted to 0.5×10⁶ cells/ml and partitioned into two treatment conditions:
Condition 1: 10 nM Rapamycin in RPMI complete
Condition 2: RPMI complete with IL2 (no Rapamycin)

On Day 14, the cells were diluted by 50% in their respective medias.

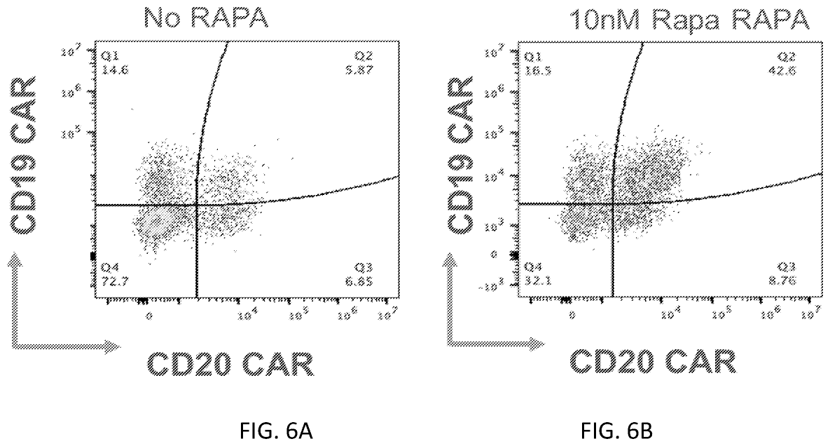

On Day 20, the T cells were stained and analyzed by flow cytometry for expression of both CD19 and CD20 CARS (FIGS. 6A and 6B). The flow cytometry analysis comprised 200K cells/sample (approximately 200 ul/sample) from three samples:
1) 0 MOI
2) 12.5 MOI
3) 12.5 MOI+Rapamycin

As compared to dual vector system transduced T cells not treated with rapamycin (5.87%), dual vector system transduced T cells demonstrate enriched expression of both CD19 and CD20 CARs following rapamycin addition (42.6%).

### Staining Procedure

The following fluorophores were used in flow cytometry analysis:
a. CD19-FITC (surface antigen)
b. CD20-PE conjugate (surface antigen)
c. DAPI (live/dead)

Cells were spun down, pseudo-washed once in PBS and then washed in PBS. For surface antigen staining, cells were suspended in MACS/0.5% BSA (“FACS”) with staining reagents as above. Cells were then pseudo-washed in FACS, then washed with FACS and re-suspended in Fluoro Fix fixative (Biolegend). Flow Cytometry analysis was performed using Cytoflex S (Beckman Coulter) using channels (Violet, Blue, Yellow, Red). Single stains and Fluorescence Minus One Control (FMO control) was performed using cells from Sample 3 (12.5 MOI+rapamycin).

### Example 3: Dual CAR T Cells Kill Target Cells

In order to assess the ability of CD19/CD20 dual CAR T cell exposure to kill CD19+ and/or CD20+ target cells, a co-culture plate was set up according the Table 3.

TABLE 3

| Effector | Target | Effector | Target |
|---|---|---|---|
| MOI 0 | none | MOI 12.5R | none |
| MOI 0 | RAJI (CD19 + CD20) | MOI 12.5R | RAJI (CD19 + CD20) |
| MOI 0 | RAJI 19 KO | MOI 12.5R | RAJI 19 KO |
| | (CD20 only) | | (CD20 only) |
| MOI 0 | K562 (no | MOI 12.5R | K562 (no |
| | antigen target) | | antigen target) |
| MOI 0 | K562 KI (CD19 only) | MOI 12.5R | K562 KI (CD19 only) |
| | RAJI alone | | K562 alone |
| | RAJI 19 KO alone | | K562 KI alone |

200,000 transduced T cells were co-cultured with 40,000 target cells in a 96 well non-treated u-bottom plate in RPMI media with 10% FBS and Penicillin/Streptomycin at 37° C. and 5% CO₂. As a control, target cells: RAJI, RAJI K562, and K562 KI were cultured alone. The cells were co-cultured for 60 hours.

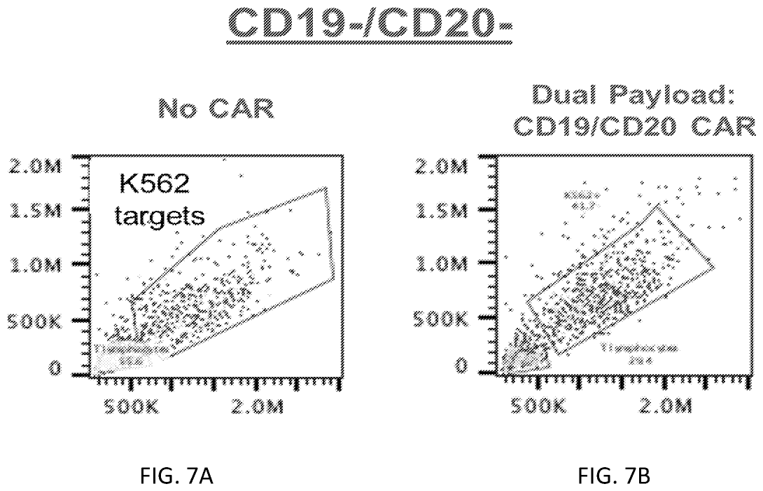

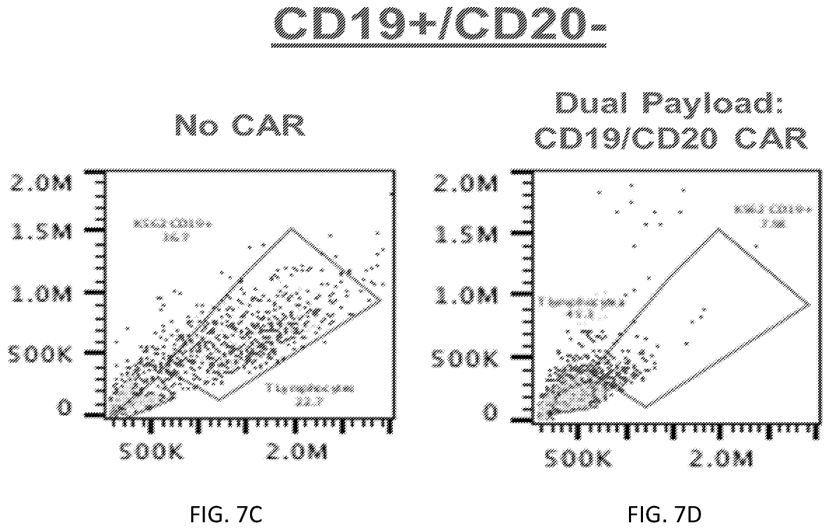

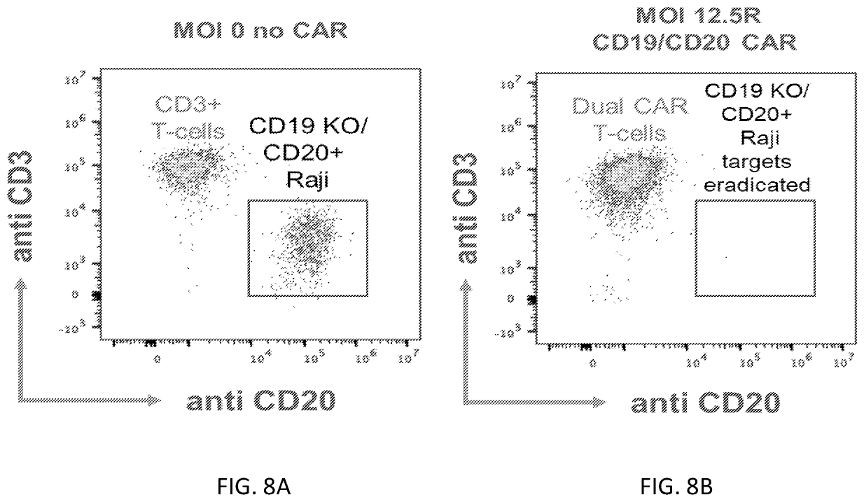

After 60 hours, the T cells were stained and analyzed by flow cytometry for analysis of target cell elimination (FIGS. 7 and 8).

The following fluorophores were used in flow cytometry analysis:
a. anti-CD3-FITC (CAR T cells)
b. anti-CD19-APC (Raji cells and K562 KI cells expressing CD19)
c. CD20-APC-Cy7 (Raji cells)

Dual vector system transduced T cells eradicated CD19 positive/CD20 negative tumor cells (FIGS. 7C-7D), while CD19 negative/CD20 negative tumors remained unaffected by the dual vector system CARS (FIGS. 7A-7B). This data affirms that the CD19 CAR expressed on dual vector system transduced T cells is functional and generates potent tumor elimination.

Dual vector system transduced T cells eradicated CD19 negative/CD20 positive tumor cells (FIGS. 8A-8B). This data affirms that the CD20 CAR expressed on dual vector system transduced T cells is functional and generates potent tumor elimination.

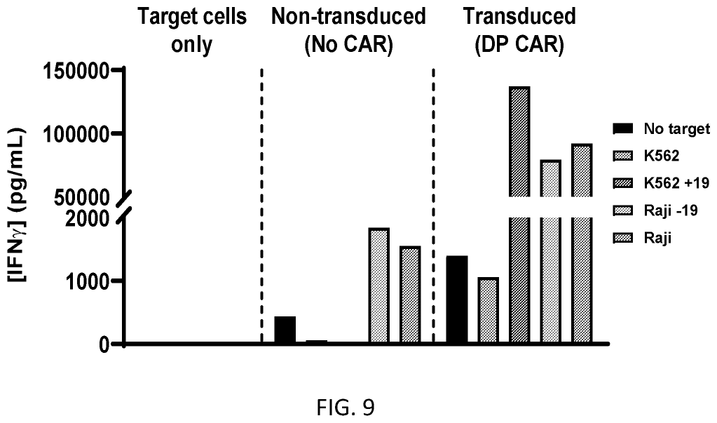

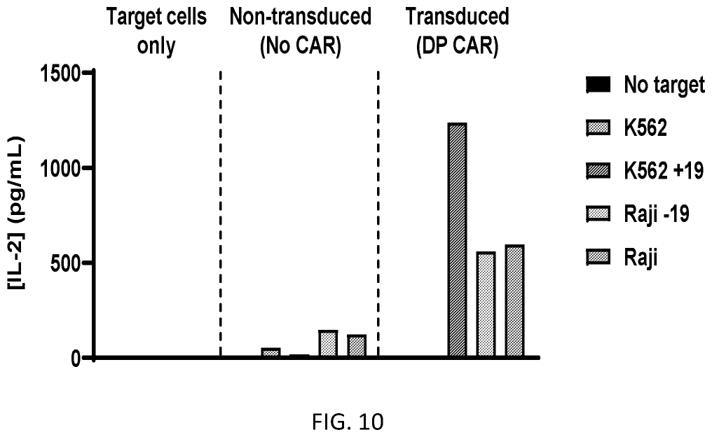

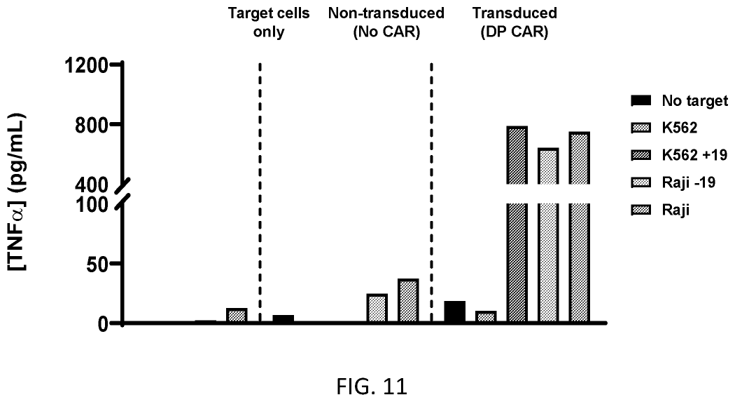

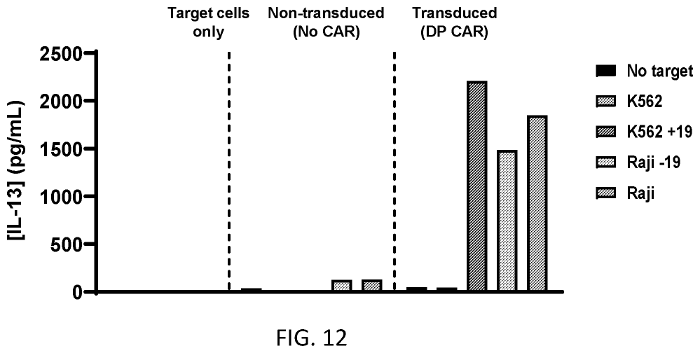

Cytokine analysis was performed for INFγ (FIG. 9), IL-2 (FIG. 10), TNFα (FIG. 11), and IL-13 (FIG. 12). Cytokine production increased in response to antigen stimulation in dual vector system transduced T cells. Target cells alone and non-transduced cells (cells lacking the CARS) did not produce cytokines.

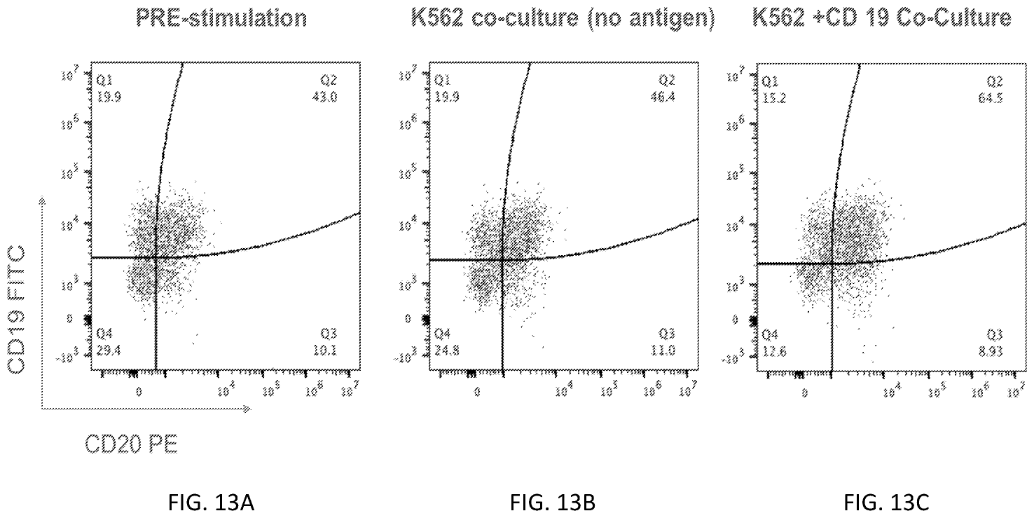

In order to assess the effect of rapamycin selection on dual CAR T cell enrichment, the 12.5 MOI+Rapamycin sample (sample 3) was analyzed by flow cytometry for surface expression of both CARS using FITC-CD19 antigen and PE-CD20 antigen as described above. The expression of both CD19 and CD20 CARs was analyzed pre-stimulation (FIG. 13A), following co-culture with K562 cells not expressing antigen (FIG. 13B), and following co-culture with K562 cells expressing CD19 (FIG. 13C). Rapamycin selection resulted in the enrichment of T cells expressing both CD19 and CD20 CARs (64.5%) as compared to pre-stimulation T cells (43.0%).

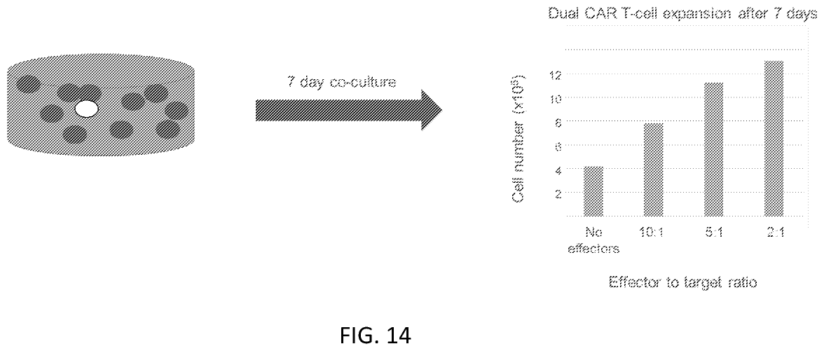

The expansion of dual vector system transduced T cells was analyzed in response target cell co-culture (FIG. 14). 1×10⁶ dual vector system transduced T cells were kept constant and plated with varying ratios of RAJI target cells in RPMI complete media with 10 nM Rapamycin in a 6 well flat-bottom plate in a total volume 3 ml/well. Cells were plated with RAJI target cells alone, 10:1, 5:1, or 2:1 (transduced effector T cell: RAJI target cell) ratios. Cells were co-cultured for 7 days and subsequently analyzed by flow cytometry for surface expression of both CARs using the FITC-CD19 antigen and the PE-CD20 antigen as described above. Cell counts were performed using viaCell. Dual vector system transduced T cells were shown to expand in response to the presence of target tumor cells containing CD19 and CD20 surface antigens (FIG. 14).

1. A vector system, comprising at least two polynucleotides, each polynucleotide comprising a polynucleotide sequence encoding a polypeptide component of a macromolecular complex,
wherein assembly of the macromolecular complex in a cell transduced with the at least two polynucleotides promotes growth and/or survival of a cell.

2. The vector system of claim 2, wherein the macromolecular complex is a multipartite cell-surface receptor.

3. The vector system of claim 1 or claim 2, wherein the vector system comprises a single vector comprising two of the polynucleotides.

4. The vector system of claim 3, wherein the single vector is a single lentivirus vector.

5. The vector system of claim 1 or claim 2, wherein the vector system comprises two vectors, each vector comprising one of the polynucleotides.

6. The vector system of claim 5, wherein the vectors are two lentivirus vectors.

7. The vector system of any one of claims 1-6, wherein assembly of the macromolecular complex is controlled by a ligand.

8. The vector system of claim 7, wherein the vector system comprises a first polynucleotide comprising a polynucleotide sequence encoding a first polypeptide component of the macromolecular complex comprising an FKBP-rapamycin complex binding domain (FRB domain) or a functional variant thereof, and a second polynucleotide comprising a polynucleotide sequence encoding a second polypeptide component of the macromolecular complex comprising an FK506 binding protein domain (FKBP) or a functional variant thereof; and/or wherein the ligand is rapamycin.

9. The vector system of claim 8, wherein the FRB domain polypeptide shares at least 75%, at least 80%, at least 85%, at least 90%, at least 95%, at least 99%, or 100% identity to SEQ ID NO: 1.

10. The vector system of claim 8, wherein the FKBP polypeptide shares at least 75%, at least 80%, at least 85%, at least 90%, at least 95%, at least 99%, or 100% identity to SEQ ID NO: 2.

11. The vector system of any one of claims 1-10, wherein expression of the macromolecular complex is under the control of an inducible genetic or biochemical system.

12. The vector system of any one of claims 1-10, wherein each polynucleotide is operatively linked to a promoter.

13. The vector system of claim 12, wherein the promoter is an inducible promoter.

14. The vector system of any one of claims 1-13, wherein at least one of the polynucleotides comprises a polynucleotide sequence that confers resistance to an immunosuppressive agent.

15. The vector system of claim 14, wherein the polynucleotide sequence that confers resistance to an immunosuppressive agent encodes a polypeptide that binds rapamycin, wherein optionally, the polypeptide is FRB.

16. The vector system of any one of claims 1-15, wherein the at least one polynucleotide sequence is capable of transducing T cells, NK cells, or NKT cells.

17. The vector system of any one of claims 1-16, wherein the at least one polynucleotide sequence is capable of transducing T cells, NK cells, or NKT cells in vivo.

18. The vector system of any one of claims 1-16, wherein the at least one polynucleotide sequence is capable of transducing T cells, NK cells, or NKT cells in vitro.

19. The vector system of any one of claims 1-18 comprising at least one retroviral particle,
wherein the retroviral particle comprises one or more transduction enhancers,
wherein the transduction enhancer is selected from the group consisting of a T-cell activation receptor, a NK-cell activation receptor, and a co-stimulatory molecule.

20. The vector system of claim 19, wherein the one or more transduction enhancers comprise one or more of anti-CD3scFv, CD86, and CD137L.

21. The vector system of any one of claims 1-20, wherein the first vector comprises a polynucleotide sequence encoding:
(a) a promoter;
(b) a FK506 binding protein (FKBP) domain or a portion thereof
(c) an 1L-2 receptor transmembrane domain
(d) an interleukin-2 receptor subunit gamma (IL2Rγ) domain; and
(e) a first chimeric antigen receptor (CAR).

22. The vector system of any one of claims 1-21, wherein the second vector comprises a polynucleotide sequence encoding:
(a) a promoter;
(b) FKBP rapamycin binding (FRB) domain or a portion thereof
(c) an it-2 receptor transmembrane domain
(d) an interleukin-2 receptor subunit beta (IL2Rβ) domain; and
(e) a second CAR.

23. The vector system of claim 21 or 22, wherein the FKBP domain or a portion thereof and FRB domain or a portion thereof heterodimerize in the presence of rapamycin to promote growth and/or survival of a cell.

24. The vector system of any one of claims 1-23, wherein the promoter is MND.

25. The vector system of claim 24, wherein the MND promoter shares at least 75%, at least 80%, at least 85%, at least 90%, at least 95%, at least 99%, or 100% identity to SEQ ID NO: 3.

26. The vector system of claim 21, wherein the IL2Rγ domain polypeptide shares at least 75%, at least 80%, at least 85%, at least 90%, at least 95%, at least 99%, or 100% identity to SEQ ID NO: 4.

27. The vector system of claim 22, wherein the IL2Rβ domain polypeptide shares at least 75%, at least 80%, at least 85%, at least 90%, at least 95%, at least 99%, or 100% identity to SEQ ID NO: 5.

28. The vector system of any one of claims 21-27, wherein the first CAR polypeptide comprises an antigen binding molecule that specifically binds to the cell surface antigen CD19.

29. The vector system of any one of claims 21-27, wherein the second CAR polypeptide comprises an antigen binding molecule that specifically binds to the cell surface antigen CD20.

30. A method, comprising:
administering to a subject a vector system of any of claims 1-29.
